SEQUENCE LISTING (168 sequences)

SEQ ID NO: 1 - protein, 17 aa, Vesicular stomatitis virus
MKCLLYLAFLFIGVNCK

SEQ ID NO: 2 - protein, 42 aa, synthetic construct
FEHPHIQDAASQLPDDESLFFGDTGLSKNPIELVEGWFSSWK

SEQ ID NO: 3 - protein, 49 aa, synthetic construct
SSIASFFFIIGLIIGLFLVLRVGIHLCIKLKHTKKRQIYTDIEMNRLGK

SEQ ID NO: 4 - protein, 23 aa, synthetic construct
FFGDTGLSKNPIELVEGWFSSWK

SEQ ID NO: 5 - protein, 24 aa, Vesicular stomatitis virus
FFGDTGLSKNPIELVEGWFSSWKS

SEQ ID NO: 6 - protein, 23 aa, Vesicular stomatitis virus
FFFIIGLIIGLFLVLRVGIHLCI

SEQ ID NO: 7 - protein, 21 aa, Vesicular stomatitis virus
KLKHTKKRQIYTDIEMNRLGK

SEQ ID NO: 8 - protein, 108 aa, synthetic construct
MKCLLYLAFLFIGVNCKFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 9 - protein, 108 aa, synthetic construct
MKCLLYLAFLFIGVNCKFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 10 - protein, 107 aa, synthetic construct
MKCLLYLAFLFIGVNCKFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 11 - protein, 110 aa, synthetic construct
MKCLLYLAFLFIGVNCKWEHPHIEAAQTFLKKDDTGEVLYYGDTGVSKNPVELVEGWFSGWRSSLMGVLAVIIGFVILMFLIKLIGVLSSLFRPKRRPIYKSDVEMAHFR

SEQ ID NO: 12 - protein, 108 aa, synthetic construct
MKCLLYLAFLFIGVNCKFEHPHIQDAASQLPDDETLFFGDTGLSKNPIEFVEGWFSSWKSSIASFFFIIGLIIGLFLVLRVGIYLCIKLKHTKKRQIYTDIEMNRLGK

SEQ ID NO: 13 - protein, 121 aa, synthetic construct
MKCLLYLAFLFIGVNCKIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 14 - protein, 109 aa, synthetic construct
MKCLLYLAFLFIGVNCKFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 15 - protein, 118 aa, synthetic construct
MKCLLYLAFLFIGVNCKLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 16 - protein, 112 aa, synthetic construct
MKCLLYLAFLFIGVNCKVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 17 - protein, 108 aa, synthetic construct
MKCLLYLAFLFIGVNCKFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 18 - protein, 111 aa, synthetic construct
MKCLLYLAFLFIGVNCKIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 19 - protein, 147 aa, synthetic construct
MKCLLYLAFLFIGVNCKIDHPQKAIASVHLNTDEELFFGNTGSDSNPVEAVEGWFASWKSAGINMALIVLCVLLVLIFLRSLPALIKLIHRYRVSRSRQTDVELNSINETARTGSVGPDIIPGAWRVHDSGVRQSQFFRNNPRRLGP

SEQ ID NO: 20 - protein, 113 aa, synthetic construct
MKCLLYLAFLFIGVNCKIDHPQRAHAQAVLGDEETLFFGDTGVGKNPVELITGWFSGWKETIMAVVAIFLLVIVLYGVLRCCPTICVLCKRKSRHRTKDMEMQYIPNNQRHWR

SEQ ID NO: 21 - protein, 111 aa, synthetic construct
MKCLLYLAFLFIGVNCKMDHPHLVHAKKYVSEDDEIYFGDTGVSHNPVEIFSGWFTNWKEGLMKFSILVLSILIFYVVIRLVMCIPLKCKKERKPRLEFELQPREWEYSRA

SEQ ID NO: 22 - protein, 114 aa, synthetic construct
MKCLLYLAFLFIGVNCKIDHPQIPDASGILPNSEQVYYGDTGVSKNPIELIEGWFANWKETVMSIVGLVLLITIVFTVLKCIGTCRSLRRKRKIEKDIELQEIGPYQPTTYRPR

SEQ ID NO: 23 - protein, 109 aa, synthetic construct
MKCLLYLAFLFIGVNCKFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 24 - protein, 94 aa, synthetic construct
  VARIANT 1: note = A or absent
  VARIANT 2: note = E or absent
  VARIANT 3: note = V or absent
  VARIANT 10: note = A or Q
  VARIANT 11: note = E or D
  VARIANT 13: note = P or A
  VARIANT 14: note = K or S
  VARIANT 18: note = E or D
  VARIANT 19: note = E or D
  VARIANT 21: note = T or S
  VARIANT 34: note = V or I
  VARIANT 37: note = I or V
  VARIANT 47: note = T or S
  VARIANT 48: note = V or I
  VARIANT 49: note = V or A
  VARIANT 50: note = T or S
  VARIANT 54: note = A or I
  VARIANT 57: note = V or L
  VARIANT 58: note = F or I
  VARIANT 60: note = L or G
  VARIANT 62: note = Y or F
  VARIANT 63: note = V or L
  VARIANT 65: note = A or L
  VARIANT 67: note = I or V
  VARIANT 68: note = V or G
  VARIANT 70: note = A or H
  VARIANT 71: note = V or L
  VARIANT 72: note = R or C
  VARIANT 73: note = Y or I
  VARIANT 74: note = R or K
  VARIANT 75: note = Y or L
  VARIANT 76: note = Q or K
  VARIANT 77: note = G or H
  VARIANT 78: note = S or T
  VARIANT 79: note = N or K
  VARIANT 80: note = N or K
  VARIANT 81: note = K or R
  VARIANT 82: note = R or Q
  VARIANT 85: note = N or T
  VARIANT 90: note = S or N
  VARIANT 92: note = F or L
  VARIANT 93: note = R or G
XXXFEHPHJXXAXXQLPXXEXLFFGDTGJSKNPXELXEGWFSSWKSXXXXFFFXIGXXIXLXXVXRXXIXXXXXXXXXXXXXIYXDIEMXRXXK

SEQ ID NO: 25 - protein, 94 aa, synthetic construct
AEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 26 - protein, 91 aa, synthetic construct
FEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 27 - protein, 511 aa, Vesicular stomatitis virus
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - protein, 16 aa, Vesicular stomatitis virus
MKCLLYLAFLFIGVNC

SEQ ID NO: 29 - protein, 25 aa, Homo sapiens
MGVLLTQRTLLSLVLALLFPSMASM

SEQ ID NO: 30 - protein, 19 aa, Homo sapiens
MGWSCIILFLVATATGVHS

SEQ ID NO: 31 - protein, 21 aa, Homo sapiens
MWWRLWWLLLLLLLLWPMVWA

SEQ ID NO: 32 - protein, 22 aa, Homo sapiens
MDMRVPAQLLGLLLLWLRGARC

SEQ ID NO: 33 - protein, 16 aa, Homo sapiens
MPLLLLLPLLWAGALA

SEQ ID NO: 34 - protein, 23 aa, Homo sapiens
MDAMKRGLCCVLLLCGAVFVSPS

SEQ ID NO: 35 - protein, 18 aa, Homo sapiens
MAFLWLLSCWALLGTTFG

SEQ ID NO: 36 - protein, 15 aa, Homo sapiens
MNLLLILTFVAAAVA

SEQ ID NO: 37 - protein, 20 aa, Homo sapiens
MYRMQLLSCIALSLALVTNS

SEQ ID NO: 38 - protein, 16 aa, Homo sapiens
MKWVTFISLLFSSAYS

SEQ ID NO: 39 - protein, 24 aa, Homo sapiens
MALWMRLLPLLALLALWGPDPAAA

SEQ ID NO: 40 - protein, 24 aa, Homo sapiens
MPSSVSWGILLLAGLCCLVPVSLA

SEQ ID NO: 41 - protein, 108 aa, synthetic construct; identical to SEQ ID NO: 8

SEQ ID NO: 42 - protein, 89 aa, synthetic construct
MKCLLYLAFLFIGVNCKFFGDTGLSKNPIELVEGWFSSWKSSIASFFFIIGLIIGLFLVLRVGIHLCIKLKHTKKRQIYTDIEMNRLGK

SEQ ID NO: 43 - protein, 81 aa, synthetic construct
MKCLLYLAFLFIGVNCKKNPIELVEGWFSSWKSSIASFFFIIGLIIGLFLVLRVGIHLCIKLKHTKKRQIYTDIEMNRLGK

SEQ ID NO: 44 - protein, 69 aa, synthetic construct
MKCLLYLAFLFIGVNCKSWKSSIASFFFIIGLIIGLFLVLRVGIHLCIKLKHTKKRQIYTDIEMNRLGK

SEQ ID NO: 45 - protein, 108 aa, synthetic construct; identical to SEQ ID NO: 9

SEQ ID NO: 46 - protein, 89 aa, synthetic construct
MKCLLYLAFLFIGVNCKFFGDTGISKNPVELIEGWFSSWKSTVVTFFFAIGVFILLYVVARIVIAVRYRYQGSNNKRIYNDIEMSRFRK

SEQ ID NO: 47 - protein, 81 aa, synthetic construct
MKCLLYLAFLFIGVNCKKNPVELIEGWFSSWKSTVVTFFFAIGVFILLYVVARIVIAVRYRYQGSNNKRIYNDIEMSRFRK

SEQ ID NO: 48 - protein, 107 aa, synthetic construct; identical to SEQ ID NO: 10

SEQ ID NO: 49 - protein, 88 aa, synthetic construct
MKCLLYLAFLFIGVNCKFFGDTGISKNPVEVIEGWFSNWRSSVMAIVFAILLLVITVLMVRLCVAFRHFCCQKRHKIYNDLEMNQLRR

SEQ ID NO: 50 - protein, 80 aa, synthetic construct
MKCLLYLAFLFIGVNCKKNPVEVIEGWFSNWRSSVMAIVFAILLLVITVLMVRLCVAFRHFCCQKRHKIYNDLEMNQLRR

SEQ ID NO: 51 - protein, 110 aa, synthetic construct; identical to SEQ ID NO: 11

SEQ ID NO: 52 - protein, 88 aa, synthetic construct
MKCLLYLAFLFIGVNCKYYGDTGVSKNPVELVEGWFSGWRSSLMGVLAVIIGFVILMFLIKLIGVLSSLFRPKRRPIYKSDVEMAHFR

SEQ ID NO: 53 - protein, 80 aa, synthetic construct
MKCLLYLAFLFIGVNCKKNPVELVEGWFSGWRSSLMGVLAVIIGFVILMFLIKLIGVLSSLFRPKRRPIYKSDVEMAHFR

SEQ ID NO: 54 - protein, 108 aa, synthetic construct; identical to SEQ ID NO: 12

SEQ ID NO: 55 - protein, 89 aa, synthetic construct
MKCLLYLAFLFIGVNCKFFGDTGLSKNPIEFVEGWFSSWKSSIASFFFIIGLIIGLFLVLRVGIYLCIKLKHTKKRQIYTDIEMNRLGK

SEQ ID NO: 56 - protein, 81 aa, synthetic construct
MKCLLYLAFLFIGVNCKKNPIEFVEGWFSSWKSSIASFFFIIGLIIGLFLVLRVGIYLCIKLKHTKKRQIYTDIEMNRLGK

SEQ ID NO: 57 - protein, 121 aa, synthetic construct; identical to SEQ ID NO: 13

SEQ ID NO: 58 - protein, 102 aa, synthetic construct
MKCLLYLAFLFIGVNCKFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 59 - protein, 94 aa, synthetic construct
MKCLLYLAFLFIGVNCKKNPIELIQGWFSNWRESVMAIVGIVLLIVVTFLAIKTVRVLNCLWRPRKKRIVRQEVDVESRLNHFEMRGFPEYVKR

SEQ ID NO: 60 - protein, 109 aa, synthetic construct; identical to SEQ ID NO: 14

SEQ ID NO: 61 - protein, 90 aa, synthetic construct
MKCLLYLAFLFIGVNCKFFGDTGLSKNPVELVEGWFSSWKSTLASFFLIIGLGVALIFIIRIIVAIRYKYKGRKTQKIYNDVEMSRLGNK

SEQ ID NO: 62 - protein, 82 aa, synthetic construct
MKCLLYLAFLFIGVNCKKNPVELVEGWFSSWKSTLASFFLIIGLGVALIFIIRIIVAIRYKYKGRKTQKIYNDVEMSRLGNK

SEQ ID NO: 63 - protein, 118 aa, synthetic construct; identical to SEQ ID NO: 15

SEQ ID NO: 64 - protein, 99 aa, synthetic construct
MKCLLYLAFLFIGVNCKFFGDTGVSKNPVELVTGWFTSWKESLAAGVVLILVVVLIYGVLRCFPVLCTTCRKPKWKKGVERSDSFEMRIFKPNNMRARV

SEQ ID NO: 65 - protein, 91 aa, synthetic construct
MKCLLYLAFLFIGVNCKKNPVELVTGWFTSWKESLAAGVVLILVVVLIYGVLRCFPVLCTTCRKPKWKKGVERSDSFEMRIFKPNNMRARV

SEQ ID NO: 66 - protein, 112 aa, synthetic construct; identical to SEQ ID NO: 16

SEQ ID NO: 67 - protein, 93 aa, synthetic construct
MKCLLYLAFLFIGVNCKFFGDTGVSKNPIELISGWFSDWKETAAALGFAAISVILIIGLMRLLPLLCRRRKQKKVIYKDVELNSFDPRQAFHR

SEQ ID NO: 68 - protein, 85 aa, synthetic construct
MKCLLYLAFLFIGVNCKKNPIELISGWFSDWKETAAALGFAAISVILIIGLMRLLPLLCRRRKQKKVIYKDVELNSFDPRQAFHR

SEQ ID NO: 69 - protein, 108 aa, synthetic construct; identical to SEQ ID NO: 17

SEQ ID NO: 70 - protein, 89 aa, synthetic construct
MKCLLYLAFLFIGVNCKFFGDTGLSKNPIDFVEGWFSSWKSSIASFFFIIGLIIGLFLVLRVGIYLYIKLKHTKKRQIYTDIEMNRLGR

SEQ ID NO: 71 - protein, 81 aa, synthetic construct
MKCLLYLAFLFIGVNCKKNPIDFVEGWFSSWKSSIASFFFIIGLIIGLFLVLRVGIYLYIKLKHTKKRQIYTDIEMNRLGR

SEQ ID NO: 72 - protein, 111 aa, synthetic construct; identical to SEQ ID NO: 18

SEQ ID NO: 73 - protein, 92 aa, synthetic construct
MKCLLYLAFLFIGVNCKFFGDTGVSKNPVEVVEGWFSGWRSSLMSIFGIILLIVCLVLIVRILIALKYCCVRHKKRTIYKEDLEMGRIPRRA

SEQ ID NO: 74 - protein, 84 aa, synthetic construct
MKCLLYLAFLFIGVNCKKNPVEVVEGWFSGWRSSLMSIFGIILLIVCLVLIVRILIALKYCCVRHKKRTIYKEDLEMGRIPRRA

SEQ ID NO: 75 - protein, 147 aa, synthetic construct; identical to SEQ ID NO: 19

SEQ ID NO: 76 - protein, 128 aa, synthetic construct
MKCLLYLAFLFIGVNCKFFGNTGSDSNPVEAVEGWFASWKSAGINMALIVLCVLLVLIFLRSLPALIKLIHRYRVSRSRQTDVELNSINETARTGSVGPDIIPGAWRVHDSGVRQSQFFRNNPRRLGP

SEQ ID NO: 77 - protein, 120 aa, synthetic construct
MKCLLYLAFLFIGVNCKSNPVEAVEGWFASWKSAGINMALIVLCVLLVLIFLRSLPALIKLIHRYRVSRSRQTDVELNSINETARTGSVGPDIIPGAWRVHDSGVRQSQFFRNNPRRLGP

SEQ ID NO: 78 - protein, 113 aa, synthetic construct; identical to SEQ ID NO: 20

SEQ ID NO: 79 - protein, 94 aa, synthetic construct
MKCLLYLAFLFIGVNCKFFGDTGVGKNPVELITGWFSGWKETIMAVVAIFLLVIVLYGVLRCCPTICVLCKRKSRHRTKDMEMQYIPNNQRHWR

SEQ ID NO: 80 - protein, 86 aa, synthetic construct
MKCLLYLAFLFIGVNCKKNPVELITGWFSGWKETIMAVVAIFLLVIVLYGVLRCCPTICVLCKRKSRHRTKDMEMQYIPNNQRHWR

SEQ ID NO: 81 - protein, 111 aa, synthetic construct; identical to SEQ ID NO: 21

SEQ ID NO: 82 - protein, 92 aa, synthetic construct
MKCLLYLAFLFIGVNCKYFGDTGVSHNPVEIFSGWFTNWKEGLMKFSILVLSILIFYVVIRLVMCIPLKCKKERKPRLEFELQPREWEYSRA

SEQ ID NO: 83 - protein, 84 aa, synthetic construct
MKCLLYLAFLFIGVNCKHNPVEIFSGWFTNWKEGLMKFSILVLSILIFYVVIRLVMCIPLKCKKERKPRLEFELQPREWEYSRA

SEQ ID NO: 84 - protein, 114 aa, synthetic construct; identical to SEQ ID NO: 22

SEQ ID NO: 85 - protein, 95 aa, synthetic construct
MKCLLYLAFLFIGVNCKYYGDTGVSKNPIELIEGWFANWKETVMSIVGLVLLITIVFTVLKCIGTCRSLRRKRKIEKDIELQEIGPYQPTTYRPR

SEQ ID NO: 86 - protein, 87 aa, synthetic construct
MKCLLYLAFLFIGVNCKKNPIELIEGWFANWKETVMSIVGLVLLITIVFTVLKCIGTCRSLRRKRKIEKDIELQEIGPYQPTTYRPR

SEQ ID NO: 87 - protein, 109 aa, synthetic construct; identical to SEQ ID NO: 23

SEQ ID NO: 88 - protein, 90 aa, synthetic construct
MKCLLYLAFLFIGVNCKFFGDTGLSKNPIELVEGWFSGWKSTIASFFFIIGLVIGLYLVLRIGIALCIKCRVQEKRPKIYTDVEMNRLDR

SEQ ID NO: 89 - protein, 82 aa, synthetic construct
MKCLLYLAFLFIGVNCKKNPIELVEGWFSGWKSTIASFFFIIGLVIGLYLVLRIGIALCIKCRVQEKRPKIYTDVEMNRLDR

SEQ ID NO: 90 - protein, 1936 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEPKKKRKVGS

SEQ ID NO: 91 - protein, 1710 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 92 - protein, 198 aa, Homo sapiens [sequence omitted]

SEQ ID NO: 93 - protein, 191 aa, Homo sapiens
LMDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 94 - protein, 175 aa, Homo sapiens
MDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPL

SEQ ID NO: 95 - protein, 229 aa, Rattus norvegicus [sequence omitted]

SEQ ID NO: 96 - protein, 397 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFRPWKGLEIISRRTQRRLRRIKESWGLQDLVNDFGNLQLGPPMSN

SEQ ID NO: 97 - protein, 199 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRR

SEQ ID NO: 98 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 99 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 100 - protein, 186 aa, Homo sapiens
PPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 101 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 102 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 103 - protein, 189 aa, Homo sapiens
KEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 104 - protein, 383 aa, Escherichia coli
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 105 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 106 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 107 - protein, 1419 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGGGGSGKRTADGSEFEPKKKRKVSSGGDYKDHDGDYKDHDIDYKDDDDK

SEQ ID NO: 108 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 109 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 110 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 111 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 112 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 113 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 114 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 115 - protein, 118 aa, Homo sapiens
NPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 116 - protein, 118 aa, Homo sapiens
NPDREGWLLKLGGGRVKTWKCRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 117 - protein, 59 aa, Homo sapiens
DNEVQNCMACGKGFSVTVRRHHCRQCGNIFCAECSAKNALTPSSKKPVRVCDACFNDLQ

SEQ ID NO: 118 - protein, 59 aa, Homo sapiens
DNEVQNCMACGKGFSVTVRRHHCLQCGNIFCAECSAKNALTPSSKKPVRVCDACFNDLQ

SEQ ID NO: 119 - protein, 122 aa, synthetic construct
DVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDS

SEQ ID NO: 120 - protein, 122 aa, synthetic construct
DVAISANIADIEEKRGFTSHFVFVIEVKTKGGSKYLIYLRYRQFHALQSKLEERFGPDSKSSALACTLPTLPAKVYVGVKQEIAEMRIPALNAYMKSLLSLPVWVLMDEDVRIFFYQSPYDS

SEQ ID NO: 121 - protein, 126 aa, Homo sapiens
MQTGRTEDDLVPTAPSLGTKEGYLTKQGGLVKTWKTRWFTLHRNELKYFKDQMSPEPIRILDLTECSAVQFDYSQERVNCFCLVFPFRTFYLCAKTGVEADEWIKILRWKLSQIRKQLNQGEGTIR

SEQ ID NO: 122 - protein, 153 aa, Homo sapiens
PFKIPEDDGNDLTHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 123 - protein, 154 aa, Homo sapiens
MPFKIPEDDGNDLTHTFFNPDREGWLLKLGGRVKTWKCRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 124 - protein, 101 aa, Homo sapiens
MGSGSAREGWLFKWTNYIKGYQRRWFVLSNGLLSYYRSKAEMRHTCRGTINLATANITVEDSCNFIISNGGAQTYHLKASSEVERQRWVTALELAKAKAVK

SEQ ID NO: 125 - protein, 101 aa, Homo sapiens
MGSGSAREGWLFKWTNYIKGYQERWFVLSNGLLSYYRSKAEMRHTCRGTINLATANITVEDSCNFIISNGGAQTYHLKASSEVERQRWVTALELAKAKAVK

SEQ ID NO: 126 - protein, 177 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 127 - protein, 177 aa, Homo sapiens
MAAVILESIFLKRSQQKKKTSPLNFKKCLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 128 - protein, 98 aa, Homo sapiens
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDT

SEQ ID NO: 129 - protein, 95 aa, Homo sapiens
PVERCGVLSKWTNYIHGWQDRWVVLKNNALSYYKSEDETEYGCRGSICLSKAVITPHDFDECRFDISVNDSVWYLRAQDPDHRQQWIDAIEQHKT

SEQ ID NO: 130 - protein, 102 aa, Homo sapiens
MRIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVAS

SEQ ID NO: 131 - protein, 98 aa, Homo sapiens
MDVLKQGYMMKKGHRRKNWTERWFVLKPNIISYYVSEDLKDKKGDILLDENCCVESLPDKDGKKCLFLVKCFDKTFEISASDKKKKQEWIQAIHSTIH

SEQ ID NO: 132 - protein, 98 aa, Homo sapiens
MDVLKQGYMMKKGHEEKNWTERWFVLKPNIISYYVSEDLKDKKGDILLDENCCVESLPDKDGKKCLFLVKCFDKTFEISASDKKKKQEWIQAIHSTIH

SEQ ID NO: 133 - protein, 114 aa, Homo sapiens
MDMLSSHHYKSFKVSMIHRLRFTTDVQLGISGDKVEIDPVTNQKASTKFWIKQKPISIDSDLLCACDLAEEKSPSHAIFKLTYLSNHDYKHLYFESDAATVNEIVLKVNYILES

SEQ ID NO: 134 - protein, 122 aa, Homo sapiens
MGTVMKEGWMVHYTSKDTLRKRHYWRLDSKCITLFQNDTGSRYYKEIPLSEILSLEPVKTSALIPNGANPHCFEITTANVVYYVGENVVNPSSPSPNNSVLTSGVGADVARMWEIAIQHALM

SEQ ID NO: 135 - protein, 105 aa, Homo sapiens
FIMEGPLTRIGAKHERHIFLFDGLMISCKPNHGQTRLPGYSSAEYRLKEKFVMRKIQICDKEDTCEHKHAFELVSKDENSIIFAAKSAEEKNNWMAALISLHYRS

SEQ ID NO: 136 - protein, 107 aa, Homo sapiens
QGTNLPPSRQIVIRKGWLTISNIGIMKGGSKGYWFVLTAESLSWYKDDEEKEKKYMLPLDNLKVRDVEKSFMSSKHIFALFNTEQRNVYKDYRFLELACDSQEDVDS

SEQ ID NO: 137 - protein, 159 aa, Homo sapiens
QLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQK

SEQ ID NO: 138 - protein, 117 aa, Homo sapiens
KLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEIRKVLT

SEQ ID NO: 139 - protein, 175 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKELNIQ

SEQ ID NO: 140 - protein, 170 aa, Homo sapiens
MDSGRDFLTLHGLQDDEDLQALLKGSQLLKVKSTSWRRELFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLK

SEQ ID NO: 141 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 142 - protein, 168 aa, Homo sapiens
MSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 143 - protein, 168 aa, Homo sapiens
MSDVAIVKEGWLHRRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 144 - protein, 168 aa, Homo sapiens
MSDVAIVREGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 145 - protein, 168 aa, Homo sapiens
MSDVAIVKEGWLHKRGEYIKTWRPRYFLLKNDGTFIGYKERPQDVDQREAPLNNFSVAQCQLMKTERPRPNAFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 146 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVEAPEEREEWTTAIQTVADGLKKQEEEEMDFRSGSPSDNSGAEEMEVSLAKPKHRVTMNEFEYLKLLGKGTFGKVDPPV

SEQ ID NO: 147 - protein, 168 aa, Homo sapiens
MSDVAIVKEGWLHKRGEYIKTWRPCYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 148 - protein, 168 aa, Homo sapiens
MSDVAIVKEGWLHKRGEYIKTWRPRYFLLKNDGDFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 149 - protein, 168 aa, Homo sapiens
MSDVAIVKEGWLHKRGEYIKTWRPRYFLLKNDGFFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 150 - protein, 168 aa, Homo sapiens
MSDVAIVKEGWLHKRGEYIKTWRPRYFLLKNDGLFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 151 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWYTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 152 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPAAAVTMNEFEYLKLLGKGTFGKVDPPV

SEQ ID NO: 153 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTCAIQTVADGLKKQEEEEMDFRSGSPSDNSGAEEMEVSLAKPKHRVTMNEFEYLKLLGKGTFGKVDPPV

SEQ ID NO: 154 - protein, 92 aa, Homo sapiens
KMGPVDKRKGLFARRRQLLLTEGPHLYYVDPVNKVLKGEIPWSQELRPEAKNFKTFFVHTPNRTYYLMDPSGNAHKWCRKIQEVWRQRYQSH

SEQ ID NO: 155 - protein, 130 aa, unidentified
MASNFTQFVLVDNGGTGDVTVAPSNFANGIAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 156 - protein, 62 aa, synthetic construct
MKSIRCKNCNKLLFKADSFDHIEIRCPRCKRHIIMLNACEHPTEKHCGKREKITHSDETVRY

SEQ ID NO: 157 - protein, 131 aa, unidentified
MAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLE

SEQ ID NO: 158 - protein, 102 aa, synthetic construct
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFV

SEQ ID NO: 159 - protein, 74 aa, Homo sapiens
MADFETDESVLMRRQKQINYGKNTIAYDRYIKEVPRHLRQPGIHPKTPNKFKKYSRRSWDQQIKLWKVALHFWD

SEQ ID NO: 160 - protein, 38 aa, Herpes simplex virus
PTDALDDFDLDMLPADALDDFDLDMLPADALDDFDLDM

SEQ ID NO: 161 - protein, 53 aa, synthetic construct
GRADALDDFDLDMLGSDALDDFDLDMLGSDALDDFDLDMLGSDALDDFDLDML

SEQ ID NO: 162 - protein, 261 aa, Homo sapiens
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 163 - protein, 190 aa, unidentified
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 164 - protein, 72 aa, Homo sapiens
MDAKSLTAWSRTLVTFKDVFVDFTREEWKLLDTAQQIVYRNVMLENYKNLVSLGYQLTKPDVILRLEKGEEP

SEQ ID NO: 165 - protein, 289 aa, Homo sapiens
EASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 166 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 167 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 168 - protein, 546 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

(19) United States
(12) Patent Application Publication
(10) Pub. No.: US 2024/0191208 A1
(43) Pub. Date: Jun. 13, 2024

(54) Minimal Virus-Like Particles and Methods of Use Thereof for Delivery of Biomolecules

(71) Applicant: The General Hospital Corporation, Boston, MA (US)

(72) Inventors: J. Keith Joung, Winchester, MA (US); Peter Cabeceiras, Boston, MA (US)

(21) Appl. No.: 18/511,459

(22) Filed: Nov. 16, 2023

Related U.S. Application Data
(60) Provisional application No. 63/425,890, filed on Nov. 16, 2022.

(51) Int. Cl. C12N 7/00; C07K 14/005; C12N 5/071; C12N 5/0783; C12N 9/22; C12N 15/11; C12N 15/86

(52) U.S. Cl. CPC C12N 7/00; C07K 14/005; C12N 5/0636; C12N 5/0686; C12N 9/22; C12N 15/11; C12N 15/86; C12N 2310/20; C12N 2760/20222; C12N 2760/20223

(57) ABSTRACT

Described herein are minimal virus-like particles (mVLPs), comprising a membrane comprising a phospholipid bilayer with one or more ectodomain-truncated VSV envelope glycoproteins on the external side; and a biomolecule cargo disposed in the core of the mVLP on the inside of the membrane. Preferably, the mVLPs do not comprise a protein from viral gag, pro, pol, or other viral proteins that reside inside of enveloped particles. Also described are methods of use of the mVLPs for delivery of the biomolecule cargo to cells.

### CLAIM OF PRIORITY

This application claims the benefit of U.S. Provisional Patent Application Ser. No. 63/425,890, filed on Nov. 16, 2022. The entire contents of the foregoing are hereby incorporated by reference.

### FEDERALLY SPONSORED RESEARCH OR DEVELOPMENT

This invention was made with Government support under Grant No. GM118158 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on Feb. 22, 2024, is named 29539-0682001_SL.xml and is 195,405 bytes in size.

### TECHNICAL FIELD

Described herein are virus-like particles (VLPs) and minimal virus-like particles (mVLPs), comprising a membrane comprising a phospholipid bilayer with one or more ectodomain-truncated VSV envelope glycoproteins on the external side; and a biomolecule cargo disposed in the core of the VLP or mVLP on the inside of the membrane. Preferably, the mVLPs do not comprise any other exogenous virally derived proteins, e.g., proteins from viral gag, pro, or pol, or other viral proteins that reside inside of enveloped particles (unless the cargo comprises the viral protein(s)). Also described are methods of use of the VLPs or mVLPs for delivery of the biomolecule cargo to cells.

### BACKGROUND

Delivery of cargo such as proteins, nucleic acids, and/or chemicals into the cytosol of living cells has been a significant hurdle in the development of biological therapeutics.

### SUMMARY

Described herein are virus-like particles (VLPs) and minimal virus-like particles (mVLPs) that are capable of packaging and delivering a wide variety of payloads, e.g., biomolecules including nucleic acids (DNA, RNA) or proteins, chemical compounds including small molecules, and/or other molecules, and any combination thereof, into eukaryotic cells. The non-viral mVLP systems described herein have the potential to be simpler, more efficient and safer than conventional, artificially-derived lipid/gold nanoparticles and viral particle-based delivery systems, at least because mVLPs have no virus-derived components except for an ectodomain-truncated envelope glycoprotein, mVLPs can utilize but do not require chemical-based dimerizers, and mVLPs have the ability to package and deliver cargo including, but not limited to, biomolecules including nucleic acids and proteins, e.g., specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, internal ribosomal entry site containing RNA, etc.), proteins, chemical compounds and/or molecules, and combinations of the above listed cargos (e.g., AAV particles and/or ribonucleoprotein (RNP) complexes comprising RNA and protein, e.g., guide RNA/CRISPR Cas protein complexes). The virus-like particles (VLPs) described herein can comprise ectodomain-truncated envelope glycoproteins. The mVLPs described herein are different from conventional retroviral particles, virus-like particles (VLPs), exosomes and other previously described extracellular vesicles that can be loaded with cargo because of the membrane configuration, ectodomain-truncated envelope glycoprotein, vast diversity of possible cargos that are enabled by novel, innovative loading strategies, the lack of a limiting DNA/RNA length constraint, the lack of proteins derived from any viral gag, pro, or pol, and the mechanism of cellular entry.

Provided herein are truncated glycoproteins/envelope proteins (tENV) comprising: an N-terminal portion comprising a signal sequence, optionally comprising the MKCLLYLAFLFIGVNCK (SEQ ID NO:1) fused to a central portion comprising all or part of a GS domain from at least one vesiculovirus G protein, optionally a VSV-G protein or homolog, ortholog, or paralog thereof, optionally comprising the sequence FEHPHIQDAASQLPDDESLFFGDTGLSKNPIELVEG WFSSWK (SEQ ID NO:2), optionally with a deletion of at least one amino acid (e.g., a truncation from the N terminal end of the central portion), which is fused to a C-terminal portion comprising a transmembrane domain and an intracellular domain, optionally comprising SSIASFFFIIGLIIGLFLVLRVGIHLCIKLKHTKKRQIYTDIE MNRLGK (SEQ ID NO:3). In some embodiments, the central portion comprises a deletion of at least 1, 2, 3, 4, 5, 6, 7, 8, 9, 10, 15, 20, 25, 30, 35, 38, 39, 40, 41, or all 42 amino acids of SEQ ID NO:2 amino acids, e.g., at least about 1, 2, 3, 4, 5, 6, 7, 8 or 10 amino acids, up to about 15, 20, 35, 30, 35, 38, 39, 40, 41, or all 42 amino acids, with any range therebetween. In some embodiments, the central portion comprises or consists of FFGDTGLSKNPIELVEGWFSSWK (SEQ ID NO:4) or FEHPHIQDAASQLPDDESLFFGDTGLSKNPIELVEGWFSSWK (SEQ ID NO:2). In some embodiments, the tENV comprises a sequence that is at least 95% identical to a sequence set forth herein, e.g., in Table 1.

Also provided herein are nucleic acids encoding the tENVs, and vectors comprising the nucleic acids, optionally operably linked to a promoter for expression of the tENV, as well as host (e.g., producer) cells comprising the nucleic acids, and optionally expressing the tENV.

Further, provided herein are virus-like particles (VLPs) comprising a tENV described herein. Also provided are minimal virus-like particles (mVLPs), comprising a membrane comprising a phospholipid bilayer and a tENV as described herein; and a cargo disposed in the core of the mVLP, wherein the cargo is optionally fused to a phospholipid bilayer recruitment domain; and, wherein the mVLP does not comprise any exogenous virally derived proteins, e.g., proteins from viral gag, pro, or pol, or other viral proteins that reside inside of enveloped particles (unless the cargo comprises the viral protein(s)). In some embodiments, the VLPs may include endogenous components, e.g., from endogenous retroviral sequences that have integrated into the genome of the cells, e.g., HERVs when particles are produced in human cells. In some embodiments, the VLPs do or do not comprise any human endogenous retroviral (HERV) proteins other than the env, e.g., do not comprise gag, pol, or pro. Exogenous virally-derived gag, pol, or pro refers to any gag, pro, pol, gag-pol, gag-pro-pol, and/or pol protein, or any other protein expressed from gag, pro, or pol, from any virus introduced into the cell.

In some embodiments, the cargo is a therapeutic or diagnostic protein or nucleic acid encoding a therapeutic or diagnostic protein, or a chemical, optionally a small molecule therapeutic or diagnostic. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof, a nucleic acid encoding a zinc finger (ZF), transcription activator-like effector (TALE), and/or CRISPR-Cas protein, variant, or fusion thereof, or a ribonucleoprotein complex (RNP) comprising a CRISPR-Cas protein, variant, or fusion thereof and optionally a guide RNA. In some embodiments, the cargo is selected from the proteins listed in Tables 2, 3, 4 & 5, or that is at least 95% identical to a sequence set forth herein, e.g., in Tables 2, 3, 4, and 5. In some embodiments, the cargo comprises a CRISPR-Cas protein, and the mVLP further comprises one or more guide RNAs that bind to and direct the CRISPR-Cas protein to a target nucleic acid sequence. In some embodiments, the cargo comprises a fusion to a phospholipid bilayer recruitment domain, preferably as shown in Table 6, or that is at least 95% identical to a sequence set forth herein in Table 6.

Also provided are methods of delivering a cargo to a target cell, optionally a cell in vivo or in vitro, the method comprising contacting the cell with a VLP or mVLP as described herein comprising the cargo.

Additionally provided herein are method of producing a VLP or an mVLP, optionally comprising a cargo. The methods comprise providing a cell expressing a tENV as described herein and optionally a cargo, optionally wherein the cell does not express exogenous virally derived proteins, e.g., proteins from viral gag, pro, or pol, or other viral proteins that reside inside of enveloped particles (unless the cargo comprises the viral protein(s)); and maintaining the cell under conditions such that the cells produce the VLPs or mVLPs.

In some embodiments, the methods further comprise harvesting and optionally purifying and/or concentrating the produced VLPs or mVLPs. In some embodiments, the cargo is a therapeutic or diagnostic protein or nucleic acid encoding a therapeutic or diagnostic protein, or a small molecule, optionally a therapeutic or diagnostic small molecule. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof, a nucleic acid encoding a zinc finger (ZF), transcription activator-like effector (TALE), and/or CRISPR-Cas protein, variant, or fusion thereof, or a ribonucleoprotein complex (RNP) comprising a CRISPR-Cas protein, variant, or fusion thereof and optionally a guide RNA.

In some embodiments, the cargo reagent is selected from the proteins listed in Tables 2, 3, 4 & 5, or that is at least 95% identical to a sequence set forth herein, e.g., in Tables 2, 3, 4, and 5. In some embodiments, the cargo reagent comprises a CRISPR-Cas protein, variant, or fusion thereof and the mVLP further comprises one or more guide RNAs that bind to and direct the CRISPR-based genome editing or modulating protein to a target sequence. In some embodiments, the cargo comprises a fusion to a phospholipid bilayer recruitment domain, preferably as shown in Table 6, or that is at least 95% identical to a sequence set forth herein in Table 6.

Also provided herein are cells expressing a tENV as described herein, and a cargo, optionally wherein the cell does not express an exogenous gag, pro, or pol protein. In some embodiments, the cargo is a therapeutic or diagnostic protein or nucleic acid encoding a therapeutic or diagnostic protein, or a small molecule, optionally a therapeutic or diagnostic small molecule. In some embodiments, the cargo is a gene editing or epigenetic modulating reagent.

In some embodiments, the gene editing or epigenetic modulating reagent comprises a zinc finger (ZF), transcription activator-like effector (TALE), and/or CRISPR-Cas protein, variant, or fusion thereof, a nucleic acid encoding a zinc finger (ZF), transcription activator-like effector (TALE), and/or CRISPR-Cas protein, variant, or fusion thereof, or a ribonucleoprotein complex (RNP) comprising a CRISPR-Cas protein, variant, or fusion thereof and optionally a guide RNA. In some embodiments, the cargo reagent is selected from the proteins listed in Tables 2, 3, 4, & 5, or that is at least 95% identical to a sequence set forth herein, e.g., in Tables 2, 3, 4, and 5. In some embodiments, the gene editing or epigenetic modulating reagent comprises a CRISPR-Cas protein, and the mVLP further comprises one or more guide RNAs that bind to and direct the CRISPR-Cas protein to a target sequence. In some embodiments, the cargo comprises a fusion to a phospholipid bilayer recruitment domain, preferably as shown in Table 6, or that is at least 95% identical to a sequence set forth herein in Table 6. In some embodiments, the cells are primary or stable human cell lines. In some embodiments, the cells are Human Embryonic Kidney (HEK) 293 cells or HEK293 T cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Diagrams of (A) exemplary mVLP DNA expression constructs that can be transfected into producer cells including cargo components Cas9 and a single guide RNA (sgRNA) and the 421 VSVG truncated ENV protein (wherein VSVG amino acids 421-511 preceded by a signal sequence are present, as described in Table 1) and (B) architecture of an exemplary particle to deliver a Cas9 gene editing protein or RNP complex.

FIG. 2. mVLP-mediated delivery delivery of SpCas9 RNP. K562 cells transduced with mVLPs containing human AKT Pleckstrin homology domain fused to SpCas9 and a guide RNA targeted to VEGF site 3.1. mVLPs were pseudotyped with one of three different ectodomain-truncated versions of VSVG (448-511 (448), 440-511 (440), and 421-511 (421), wherein these aforementioned VSVG residue ranges are preceded by a signal sequence, respectively). Gene modification was measured by targeted amplicon sequencing of the intended VEGF site 3.1 on-target site using NGS.

FIG. 3. Alignment of exemplary tENV sequences. Marked domains:
MKCLLYLAFLFIGVNCK—VSVG signal sequence (SEQ ID NO:1)
FFGDTGLSKNPIELVEGWFSSWKS—VSVG GS domain (SEQ ID NO:5)
FFFIIGLIIGLFLVLRVGIHLCI—VSVG TM domain (SEQ ID NO:6)
KLKHTKKRQIYTDIEMNRLGK—VSVG Intracellular domain (SEQ ID NO:7).
   Shown in the figure are SEQ ID NOs:8-23.

FIG. 4. Graphical alignment of VSCG and Cocal glycoproteins with GS-domain, transmembrane domain (Tm), and intracellular domain (ICD) annotated. Shown in the figure are SEQ ID NOs:24-26.

FIG. 5. Exemplary editing efficiencies of eVLPs that have various PH domain-Cas9 fusion cargos packaged together with an sgRNA targeted to the VEGFs3.1 site. HEK293T cells were treated with these eVLPs that had been pseudotyped with VSVG and editing efficiency was determined by amplicon sequencing.
PKD: protein kinase D1 (PRKD1)
DAPP: dual-adaptor for phosphotyrosine and 3-phosphoinositides-1 (DAPP-1)
FAPP: four-phosphate-adaptor protein (FAPP)
OSBP: oxysterol-binding protein (OSBP)
SWAP70: switch-associated protein 70 (SWAP70)
GRP: cytohesin 3 (CYTH3, formerly GRP1)
BTK: Bruton's tyrosine kinase (Btk)
PHLPP: Pleckstrin Homology Domain Leucine-rich Repeat Protein Phosphatase (PHLPP)
AKT: AKT serine/threonine kinase 1 (AKT1)
PLC: phospholipase C delta 1 (PLC1)

FIG. 6. Exemplary gene modification efficiencies induced by eVLPs that contained various mutant PH-Cas9/sgRNA (RNF2-targeted) RNP cargos. Primary T cells were treated with eVLPs pseudotyped with either VSVG or VSVG+BaeVTRless and gene modification efficiencies (y-axis) were determined by targeted amplicon sequencing of the RNF2 on-target site in those cells.

### DETAILED DESCRIPTION

Therapeutic proteins and nucleic acids hold great promise, but for many of these large biomolecules, delivery into cells is a hurdle to clinical development. Genome editing reagents such as zinc finger nucleases (ZFNs) or RNA-guided, enzymatically active/inactive DNA binding proteins such as Cas9 have undergone rapid advancements in terms of specificity and the types of edits that can be executed, but the hurdle of safe in vivo delivery still remains an important challenge for gene editing and epigenetic editing therapies.

Virus-like particles (VLPs) have been utilized to deliver mRNA and protein cargo into the cytosol of cells.^{2,3,25-30} VLPs have emerged as an alternative delivery modality to retroviral or lentiviral particles. VLPs can be designed to lack the ability to integrate retroviral DNA, and to package and deliver combinations of protein/RNP/DNA. However, most VLPs, including recently conceived VLPs that deliver genome editing reagents known to date, utilize HIV or other virally-derived gag or gag-pol protein fusions and viral proteases to generate retroviral-like particles.^{25-27,29,30} Some VLPs containing RNA-guided nucleases (RGNs) also must package and express guide RNAs from a lentiviral DNA transcript,²⁷ and some VLPs require a viral protease in order to form functional particles and release genome editing cargo.^{25-27,29} Because this viral protease recognizes and cleaves at multiple amino acid motifs, it can cause damage to the protein cargo or potentially to other endogenous proteins in target recipient cells, which could be hazardous or create challenges for therapeutic applications. Most published VLP modalities that deliver genome editing proteins or RNPs to date exhibit low in vitro and in vivo gene modification efficiencies due to low packaging and transduction efficiency.²⁵⁻²⁷ The complex viral genomes utilized for these VLP components possess multiple reading frames and employ RNA splicing that could result in spurious fusion protein products being delivered.^{25-27,29,30} The presence of reverse transcriptase, integrase, capsid and a virally-derived envelope protein in these VLPs is not ideal for many therapeutic applications because of immunogenicity and off target concerns. In addition, most retroviral particles, such as lentiviral particles, are pseudotyped with VSVG and nearly all described VLPs that deliver genome editing reagents hitherto possess and rely upon VSVG.^{2,3,25-30}

Lentivirus and standard VLPs commonly require GAG and ENV proteins to drive particle formation via budding off of the plasma membrane of producer cells into the cell culture medium. In addition, the majority of retroviral ENV proteins require post-translational modifications in the form of proteolytic cleavage of the intracellular domain (ICD) of the ENV protein in order to activate the fusogenicity of the ENV protein; this is essential for viral infectivity. Without wishing to be bound by theory, it is believed that the ENV protein alone is responsible for mVLP particle generation and the ability of mVLPs to efficiently deliver cargo into cells. As described herein, the ENV protein on the surface can be truncated and thus lack some or all of the ectodomain (tENV), even including deletion of all or part of the GS domain previously thought to be important (see, e.g., Jeetendra et al., J. Virol. 76(23): 12300-12311 (2002); US 2010/0167377). Surprisingly, as demonstrated herein, even when up to about 90% of the ENV ectodomain was deleted, VLPs comprising the truncated proteins still function to deliver cargo.

Provided herein are virus-like particles (VLPs) and minimal virus-like particles (mVLPs), comprising a membrane comprising a phospholipid bilayer with one or more ectodomain-truncated VSV envelope glycoproteins on the external side; and a biomolecule cargo disposed in the core of the VLP or mVLP on the inside of the membrane. The biomolecule cargo can be fused to a phospholipid bilayer recruitment domain as described herein. Preferably, the mVLPs do not comprise any other exogenous virally derived proteins, e.g., proteins from viral gag, pro, or pol, or other viral proteins that reside inside of enveloped particles, such as int (unless the cargo intentionally comprises the viral protein(s)).

Truncated Envelope Proteins (tENVs)

The truncated envelope proteins (tENVs) described herein include the exemplary sequences in Table 1 that are derived from vesicular stomatitis virus (VSV) and homologs thereof. The wild type sequence for the VSV glycoprotein is:

(SEQ ID NO: 27)

| | |
|---|---|
| 1 | MKCLLYLAFL FIGVNCKFTI VFPHNQKGNW KNVPSNYHYC |
| | PSSSDLNWHN DLIGTAIQVK |
| 61 | MPKSHKAIQA DGWMCHASKW VTTCDFRWYG PKYITQSIRS |
| | FTPSVEQCKE SIEQTKQGTW |
| 121 | LNPGFPPQSC GYATVTDAEA VIVQVTPHHV LVDEYTGEWV |
| | DSQFINGKCS NYICPTVHNS |
| 181 | TTWHSDYKVK GLCDSNLISM DITFFSEDGE LSSLGKEGTG |
| | FRSNYFAYET GGKACKMQYC |
| 241 | KHWGVRLPSG VWFEMADKDL FAAARFPECP EGSSISAPSQ |
| | TSVDVSLIQD VERILDYSLC |
| 301 | QETWSKIRAG LPISPVDLSY LAPKNPGTGP AFTIINGTLK |
| | YFETRYIRVD IAAPILSRMV |
| 361 | GMISGTTTER ELWDDWAPYE DVEIGPNGVL RTSSGYKFPL |
| | YMIGHGMLDS DLHLSSKAQV |
| 421 | FEHPHIQDAA SQLPDDESLF FGDTGLSKNP IELVEGWESS |
| | WKSSIASFFF IIGLIIGLFL |
| 481 | VLRVGIHLCI KLKHTKKRQI YTDIEMNRLG K |

VSVG signal sequence
(SEQ ID NO: 1)
MKCLLYLAFLFIGVNCK
VSVG GS domain
(SEQ ID NO: 5)
FFGDTGLSKNPIELVEGWESSWKS
VSVG TM domain
(SEQ ID NO: 6)
FFFIIGLIIGLFLVLRVGIHLCI
VSVG Intracellular domain
(SEQ ID NO: 7)
KLKHTKKRQIYTDIEMNRLGK

Preferably, the tENVs comprise an N-terminal signal sequence. Exemplary signal sequences include the one from the VSV-G protein, e.g., MKCLLYLAFLFIGVNCK (SEQ ID NO:1) and/or any other secretion signal sequence that is derived from VSVG (e.g., MKCLLYLAFLFIGVNC, SEQ ID NO:28) or a homolog thereof, or from a transmembrane protein and/or a synthetic/engineered signal sequence. A number of secretory signal peptide sequences are known in the art, including human signal sequences, examples of which are shown in Table A (Table adapted from novoprolabs.com/support/articles/commonly-used-leader-peptide-sequences-forefficient-secretion-of-a-recombinant-protein-expressed-in-mammalian-cells-201804211337.html).

TABLE A
Exemplary Human Secretory
Signal Peptide Sequences

| | | SEQ |
|---|---|---|
| Human Signal | | ID |
| sequence | Sequence | NO: |
| Oncostatin M | MGVLLTQRTLLSLVLALLFPSMASM | 29 |
| IgG2 H | MGWSCIILFLVATATGVHS | 30 |
| Secrecon* | MWWRLWWLLLLLLLLWPMVWA | 31 |
| IgKVIII | MDMRVPAQLLGLLLLWLRGARC | 32 |
| CD33 | MPLLLLLPLLWAGALA | 33 |
| tPA | MDAMKRGLCCVLLLCGAVFVSPS | 34 |
| Chymotrypsinogen | MAFLWLLSCWALLGTTFG | 35 |
| trypsinogen-2 | MNLLLILTFVAAAVA | 36 |
| Interleukin 2 | MYRMQLLSCIALSLALVTNS | 37 |
| (IL-2) | | |
| Albumin (HSA) | MKWVTFISLLFSSAYS | 38 |
| insulin | MALWMRLLPLLALLALWGPDPAAA | 39 |
| alpha | MPSSVSWGILLLAGLCCLVPVSLA | 40 |
| 1-antitrypsin | | |

*Barash et al., Biochem Biophys Res Commun. 2002 Jun. 21;294(4):835-42.

In some embodiments, another signal sequence that promotes secretion is used, e.g., as described in Table 5 of U.S. Ser. No. 10/993,967; von Heijne, J Mol Biol. 1985 Jul. 5; 184(1):99-105; Kober et al., Biotechnol. Bioeng. 2013; 110: 1164-1173; Tsuchiya et al., Nucleic Acids Research Supplement No. 3 261-262 (2003).

The signal sequence is fused to a central portion comprising all or part of a GS domain from a VSV-G protein or homolog thereof. In some embodiments, the central portion comprises the sequence FEHPHIQDAASQLPDDESLFFGDTGLSKNPIELVEGWFSSWK (SEQ ID NO:2), optionally with a deletion of at least one amino acid (e.g., a truncation from the N terminal end of the central portion), and a C-terminal comprising an intracellular domain from VSVG or a homolog thereof, e.g., comprising SSIASFFFIIGLIIGLFLVLRVGIHLCIKLKHTKKRQIYTDIEMNRLGK (SEQ ID NO:3). In some embodiments, the central portion comprises a deletion of about 1, 2, 3, 4, 5, 6, 7, 8, 9, 10, 20, 25, 30, 35, 38, 39, 40, 41, or all 42 amino acids of SEQ ID NO:2, and all ranges therebetween (e.g., with the recited numbers as endpoints), e.g., at least about 1, 2, 3, 4, 5, 6, 7, 8, 9, 10, 20, 25, 30, 35, 38, 39, 40, 41, or all 42 amino acids of SEQ ID NO:2, with any range therebetween; in some embodiments, the deletion is at the N terminus. In some embodiments, the central portion comprises

(SEQ ID NO: 4)

FFGDTGLSKNPIELVEGWFSSWK
or

(SEQ ID NO: 2)

FEHPHIQDAASQLPDDESLFFGDTGLSKNPIELVEGWFSSWK.

In some embodiments, the sequence is at least 95% identical to a sequence set forth herein.

As an alternative to the VSV G protein, G protein homologs from other viruses can also be used, e.g., Cocal glycoprotein; vesicular stomatitis Alagoas glycoprotein (VSAG); Vesicular stomatitis New Jersey glycoprotein (strain Ogden subtype Concan) (VSNJG); Vesicular stomatitis Indiana virus (strain Orsay) (VSOG); Piry glycoprotein; Maraba glycoprotein; Chandipura glycoprotein; Isfahan glycoprotein; vesicular stomatitis Glasgow glycoprotein (VSGG); Carajas virus glycoprotein (CVG); Radi virus glycoprotein; Jurona glycoprotein; Malpais Spring glycoprotein; Perinet Spring glycoprotein; or Morreton glycoprotein; see, e.g., FIG. 3. The sequences can include one or more deletions in the central GS domain as noted above. In some embodiments, the sequence is at least 95% identical to a sequence set forth herein.

Exemplary tENVs are provided in Table 1.

TABLE 1
Exemplary tENVs

| | | SEQ ID |
|---|---|---|
| Source | Sequence | NO: |
| Vesicular stomatitis | MKCLLYLAFLFIGVNCKFEHPHIQDAASQLPDDE | 41. |
| virus Glycoprotein | SLFFGDTGLSKNPIELVEGWFSSWKSSIASFFFI | |
| (VSVG 421 | IGLIIGLFLVLRVGIHLCIKLKHTKKRQIYTDIE | |
| Truncation) | MNRLGK | |
| Vesicular stomatitis | MKCLLYLAFLFIGVNCKFFGDTGLSKNPIELVEG | 42. |
| virus Glycoprotein | WFSSWKSSIASFFFIIGLIIGLFLVLRVGIHLCI | |
| (VSVG 440 | KLKHTKKRQIYTDIEMNRLGK | |
| Truncation) | | |
| Vesicular stomatitis | MKCLLYLAFLFIGVNCKKNPIELVEGWFSSWKSS | 43. |
| virus Glycoprotein | IASFFFIIGLIIGLFLVLRVGIHLCIKLKHTKKR | |
| (VSVG 448 | QIYTDIEMNRLGK | |
| Truncation) | | |
| Vesicular stomatitis | MKCLLYLAFLFIGVNCKSWKSSIASFFFIIGLII | 44. |
| virus Glycoprotein | GLFLVLRVGIHLCIKLKHTKKRQIYTDIEMNRLG | |
| (VSVG 460 | K | |
| Truncation) | | |
| Cocal glycoprotein | MKCLLYLAFLFIGVNCKFEHPHLAEAPKQLPEEE | 45. |
| 422 truncation | TLFFGDTGISKNPVELIEGWFSSWKSTVVTFFFA | |
| | IGVFILLYVVARIVIAVRYRYQGSNNKRIYNDIE | |
| | MSRFRK | |
| Cocal glycoprotein | MKCLLYLAFLFIGVNCKFFGDTGISKNPVELIEG | 46. |
| 441 truncation | WFSSWKSTVVTFFFAIGVFILLYVVARIVIAVRY | |
| | RYQGSNNKRIYNDIEMSRFRK | |
| Cocal glycoprotein | MKCLLYLAFLFIGVNCKKNPVELIEGWFSSWKST | 47. |
| 449 truncation | VVTFFFAIGVFILLYVVARIVIAVRYRYQGSNNK | |
| | RIYNDIEMSRFRK | |
| vesicular stomatitis | MKCLLYLAFLFIGVNCKFHHPQIAEAVQKLPDDE | 48. |
| Alagoas | TLFFGDTGISKNPVEVIEGWFSNWRSSVMAIVFA | |
| glycoprotein | ILLLVITVLMVRLCVAFRHFCCQKRHKIYNDLEM | |
| (VSAG) 422 | NQLRR | |
| truncation | | |
| vesicular stomatitis | MKCLLYLAFLFIGVNCKFFGDTGISKNPVEVIEG | 49. |
| Alagoas | WFSNWRSSVMAIVFAILLLVITVLMVRLCVAFRH | |
| glycoprotein | FCCQKRHKIYNDLEMNQLRR | |
| (VSAG) 441 | | |
| truncation | | |
| vesicular stomatitis | MKCLLYLAFLFIGVNCKKNPVEVIEGWFSNWRSS | 50. |
| Alagoas | VMAIVFAILLLVITVLMVRLCVAFRHFCCQKRHK | |
| glycoprotein | IYNDLEMNQLRR | |
| (VSAG) 449 | | |
| truncation | | |
| Vesicular stomatitis | MKCLLYLAFLFIGVNCKWEHPHIEAAQTFLKKDD | 51. |
| New Jersey | TGEVLYYGDTGVSKNPVELVEGWFSGWRSSLMGV | |
| glycoprotein (strain | LAVIIGFVILMFLIKLIGVLSSLFRPKRRPIYKS | |
| Ogden subtype | DVEMAHFR | |
| Concan) (VSNJG) | | |
| 425 truncation | | |
| Vesicular stomatitis | MKCLLYLAFLFIGVNCKYYGDTGVSKNPVELVEG | 52. |
| New Jersey | WFSGWRSSLMGVLAVIIGFVILMFLIKLIGVLSS | |
| glycoprotein (strain | LFRPKRRPIYKSDVEMAHFR | |
| Ogden subtype | | |
| Concan) (VSNJG) | | |
| 447 truncation | | |
| Vesicular stomatitis | MKCLLYLAFLFIGVNCKKNPVELVEGWFSGWRSS | 53. |
| New Jersey | LMGVLAVIIGFVILMFLIKLIGVLSSLFRPKRRP | |
| glycoprotein (strain | IYKSDVEMAHFR | |
| Ogden subtype | | |
| Concan) (VSNJG) | | |
| 455 truncation | | |
| Vesicular stomatitis | MKCLLYLAFLFIGVNCKFEHPHIQDAASQLPDDE | 54. |
| Indiana virus (strain | TLFFGDTGLSKNPIEFVEGWFSSWKSSIASFFFI | |
| Orsay) (VSOG) 421 | IGLIIGLFLVLRVGIYLCIKLKHTKKRQIYTDIE | |
| truncation | MNRLGK | |
| Vesicular stomatitis | MKCLLYLAFLFIGVNCKFFGDTGLSKNPIEFVEG | 55. |
| Indiana virus (strain | WFSSWKSSIASFFFIIGLIIGLFLVLRVGIYLCI | |
| Orsay) (VSOG) 440 | KLKHTKKRQIYTDIEMNRLGK | |
| truncation | | |
| Vesicular stomatitis | MKCLLYLAFLFIGVNCKKNPIEFVEGWFSSWKSS | 56. |
| Indiana virus (strain | IASFFFIIGLIIGLFLVLRVGIYLCIKLKHTKKR | |
| Orsay) (VSOG) 448 | QIYTDIEMNRLGK | |
| truncation | | |
| Piry glycoprotein | MKCLLYLAFLFIGVNCKIDHPQMPDAKSVLPEDE | 57. |
| 426 truncation | EIFFGDTGVSKNPIELIQGWFSNWRESVMAIVGI | |
| | VLLIVVTFLAIKTVRVLNCLWRPRKKRIVRQEVD | |
| | VESRLNHFEMRGFPEYVKR | |
| Piry glycoprotein | MKCLLYLAFLFIGVNCKFFGDTGVSKNPIELIQG | 58. |
| 445 truncation | WFSNWRESVMAIVGIVLLIVVTFLAIKTVRVLNC | |
| | LWRPRKKRIVRQEVDVESRLNHFEMRGFPEYVKR | |
| Piry glycoprotein | MKCLLYLAFLFIGVNCKKNPIELIQGWFSNWRES | 59. |
| 453 truncation | VMAIVGIVLLIVVTFLAIKTVRVLNCLWRPRKKR | |
| | IVRQEVDVESRLNHFEMRGFPEYVKR | |
| Maraba glycoprotein | MKCLLYLAFLFIGVNCKFEHPHAKDAASQLPDDE | 60. |
| 421 truncation | TLFFGDTGLSKNPVELVEGWFSSWKSTLASFFLI | |
| | IGLGVALIFIIRIIVAIRYKYKGRKTQKIYNDVE | |
| | MSRLGNK | |
| Maraba glycoprotein | MKCLLYLAFLFIGVNCKFFGDTGLSKNPVELVEG | 61. |
| 440 truncation | WFSSWKSTLASFFLIIGLGVALIFIIRIIVAIRY | |
| | KYKGRKTQKIYNDVEMSRLGNK | |
| Maraba glycoprotein | MKCLLYLAFLFIGVNCKKNPVELVEGWFSSWKST | 62. |
| 448 truncation | LASFFLIIGLGVALIFIIRIIVAIRYKYKGRKTQ | |
| | KIYNDVEMSRLGNK | |
| Chandipura | MKCLLYLAFLFIGVNCKLDHPQLPHAQSIADDSE | 63. |
| glycoprotein 430 | EIFFGDTGVSKNPVELVTGWFTSWKESLAAGVVL | |
| truncation | ILVVVLIYGVLRCFPVLCTTCRKPKWKKGVERSD | |
| | SFEMRIFKPNNMRARV | |
| Chandipura | MKCLLYLAFLFIGVNCKFFGDTGVSKNPVELVTG | 64. |
| glycoprotein 449 | WFTSWKESLAAGVVLILVVVLIYGVLRCFPVLCT | |
| truncation | TCRKPKWKKGVERSDSFEMRIFKPNNMRARV | |
| Chandipura | MKCLLYLAFLFIGVNCKKNPVELVTGWFTSWKES | 65. |
| glycoprotein 457 | LAAGVVLILVVVLIYGVLRCFPVLCTTCRKPKWK | |
| truncation | KGVERSDSFEMRIFKPNNMRARV | |
| Isfahan glycoprotein | MKCLLYLAFLFIGVNCKVDHPHVPIAQAFVSEGE | 66. |
| 429 truncation | EVFFGDTGVSKNPIELISGWFSDWKETAAALGFA | |
| | AISVILIIGLMRLLPLLCRRRKQKKVIYKDVELN | |
| | SFDPRQAFHR | |
| Isfahan glycoprotein | MKCLLYLAFLFIGVNCKFFGDTGVSKNPIELISG | 67. |
| 448 truncation | WFSDWKETAAALGFAAISVILIIGLMRLLPLLCR | |
| | RRKQKKVIYKDVELNSFDPRQAFHR | |
| Isfahan glycoprotein | MKCLLYLAFLFIGVNCKKNPIELISGWFSDWKET | 68. |
| 456 truncation | AAALGFAAISVILIIGLMRLLPLLCRRRKQKKVI | |
| | YKDVELNSFDPRQAFHR | |
| vesicular stomatitis | MKCLLYLAFLFIGVNCKFEHPHIQDAASQLPDDE | 69. |
| Glasgow | ILFFGDTGLSKNPIDFVEGWFSSWKSSIASFFFI | |
| glycoprotein | IGLIIGLFLVLRVGIYLYIKLKHTKKRQIYTDIE | |
| (VSGG) 421 | MNRLGR | |
| truncation | | |
| vesicular stomatitis | MKCLLYLAFLFIGVNCKFFGDTGLSKNPIDFVEG | 70. |
| Glasgow | WFSSWKSSIASFFFIIGLIIGLFLVLRVGIYLYI | |
| glycoprotein | KLKHTKKRQIYTDIEMNRLGR | |
| (VSGG) 440 | | |
| truncation | | |
| vesicular stomatitis | MKCLLYLAFLFIGVNCKKNPIDFVEGWFSSWKSS | 71. |
| Glasgow | IASFFFIIGLIIGLFLVLRVGIYLYIKLKHTKKR | |
| glycoprotein | QIYTDIEMNRLGR | |
| (VSGG) 448 | | |
| truncation | | |
| Carajas virus | MKCLLYLAFLFIGVNCKIEHPHAKEAQKVVDDSE | 72. |
| glycoprotein (CVG) | VIFFGDTGVSKNPVEVVEGWFSGWRSSLMSIFGI | |
| 430 truncation | ILLIVCLVLIVRILIALKYCCVRHKKRTIYKEDL | |
| | EMGRIPRRA | |
| Carajas virus | MKCLLYLAFLFIGVNCKFFGDTGVSKNPVEVVEG | 73. |
| glycoprotein (CVG) | WFSGWRSSLMSIFGIILLIVCLVLIVRILIALKY | |
| 449 truncation | CCVRHKKRTIYKEDLEMGRIPRRA | |
| Carajas virus | MKCLLYLAFLFIGVNCKKNPVEVVEGWFSGWRSS | 74. |
| glycoprotein (CVG | LMSIFGIILLIVCLVLIVRILIALKYCCVRHKKR | |
| 457 truncation) | TIYKEDLEMGRIPRRA | |
| Radi virus | MKCLLYLAFLFIGVNCKIDHPQKAIASVHLNTDE | 75. |
| glycoprotein 427 | ELFFGNTGSDSNPVEAVEGWFASWKSAGINMALI | |
| truncation | VLCVLLVLIFLRSLPALIKLIHRYRVSRSRQTDV | |
| | ELNSINETARTGSVGPDIIPGAWRVHDSGVRQSQ | |
| | FFRNNPRRLGP | |
| Radi virus | MKCLLYLAFLFIGVNCKFFGNTGSDSNPVEAVEG | 76. |
| glycoprotein 446 | WFASWKSAGINMALIVLCVLLVLIFLRSLPALIK | |
| truncation | LIHRYRVSRSRQTDVELNSINETARTGSVGPDII | |
| | PGAWRVHDSGVRQSQFFRNNPRRLGP | |
| Radi virus | MKCLLYLAFLFIGVNCKSNPVEAVEGWFASWKSA | 77. |
| glycoprotein 454 | GINMALIVLCVLLVLIFLRSLPALIKLIHRYRVS | |
| truncation | RSRQTDVELNSINETARTGSVGPDIIPGAWRVHD | |
| | SGVRQSQFFRNNPRRLGP | |
| Jurona glycoprotein | MKCLLYLAFLFIGVNCKIDHPQRAHAQAVLGDEE | 78. |
| 429 truncation | TLFFGDTGVGKNPVELITGWFSGWKETIMAVVAI | |
| | FLLVIVLYGVLRCCPTICVLCKRKSRHRTKDMEM | |
| | QYIPNNQRHWR | |
| Jurona glycoprotein | MKCLLYLAFLFIGVNCKFFGDTGVGKNPVELITG | 79. |
| 448 truncation | WFSGWKETIMAVVAIFLLVIVLYGVLRCCPTICV | |
| | LCKRKSRHRTKDMEMQYIPNNQRHWR | |
| Jurona glycoprotein | MKCLLYLAFLFIGVNCKKNPVELITGWFSGWKET | 80. |
| 456 truncation | IMAVVAIFLLVIVLYGVLRCCPTICVLCKRKSRH | |
| | RTKDMEMQYIPNNQRHWR | |
| Malpais Spring | MKCLLYLAFLFIGVNCKMDHPHLVHAKKYVSEDD | 81. |
| glycoprotein 428 | EIYFGDTGVSHNPVEIFSGWFTNWKEGLMKFSIL | |
| truncation | VLSILIFYVVIRLVMCIPLKCKKERKPRLEFELQ | |
| | PREWEYSRA | |
| Malpais Spring | MKCLLYLAFLFIGVNCKYFGDTGVSHNPVEIFSG | 82. |
| glycoprotein 447 | WFTNWKEGLMKFSILVLSILIFYVVIRLVMCIPL | |
| truncation | KCKKERKPRLEFELQPREWEYSRA | |
| Malpais Spring | MKCLLYLAFLFIGVNCKHNPVEIFSGWFTNWKEG | 83. |
| glycoprotein 455 | LMKFSILVLSILIFYVVIRLVMCIPLKCKKERKP | |
| truncation | RLEFELQPREWEYSRA | |
| Perinet Spring | MKCLLYLAFLFIGVNCKIDHPQIPDASGILPNSE | 84. |
| glycoprotein 429 | QVYYGDTGVSKNPIELIEGWFANWKETVMSIVGL | |
| truncation | VLLITIVFTVLKCIGTCRSLRRKRKIEKDIELQE | |
| | IGPYQPTTYRPR | |
| Perinet Spring | MKCLLYLAFLFIGVNCKYYGDTGVSKNPIELIEG | 85. |
| glycoprotein 448 | WFANWKETVMSIVGLVLLITIVFTVLKCIGTCRS | |
| truncation | LRRKRKIEKDIELQEIGPYQPTTYRPR | |
| Perinet Spring | MKCLLYLAFLFIGVNCKKNPIELIEGWFANWKET | 86. |
| glycoprotein 456 | VMSIVGLVLLITIVFTVLKCIGTCRSLRRKRKIE | |
| truncation | KDIELQEIGPYQPTTYRPR | |
| Morreton | MKCLLYLAFLFIGVNCKFEHPHIQDAASQLPDDE | 87. |
| glycoprotein 422 | TLFFGDTGLSKNPIELVEGWFSGWKSTIASFFFI | |
| truncation | IGLVIGLYLVLRIGIALCIKCRVQEKRPKIYTDV | |
| | EMNRLDR | |
| Morreton | MKCLLYLAFLFIGVNCKFFGDTGLSKNPIELVEG | 88. |
| glycoprotein 441 | WFSGWKSTIASFFFIIGLVIGLYLVLRIGIALCI | |
| truncation | KCRVQEKRPKIYTDVEMNRLDR | |
| Morreton | MKCLLYLAFLFIGVNCKKNPIELVEGWFSGWKST | 89. |
| glycoprotein 449 | IASFFFIIGLVIGLYLVLRIGIALCIKCRVQEKR | |
| truncation | PKIYTDVEMNRLDR | |

#, SEQ ID NO:

In some embodiments, the tENV is used in place of an ENV protein in a standard VLP, e.g., those VLPs described in previous publications.^{29,39,40} In some embodiments, the tENV is the only virally-derived component of eVLPs, e.g., as described in WO 2022/020800 (incorporated herein by reference in its entirety); we refer to these herein as minimal VLPs (mVLPs). In some embodiments, the VLPs or mVLPs can be composed of a mixture of ectosomes and exosomes that can be separated by purification, if desired. In part because of the above mentioned design simplifications and optimizations, mVLPs are particularly suited for delivery of cargo including but not limited to DNA, RNA, protein, or combinations of biomolecules and/or chemicals, such as DNA-encoded or RNP-based genome editing reagents.

Thus described herein are various embodiments of minimal virus-like particles (mVLPs), which provide a platform for the delivery of cargo including nucleic acids and proteins, e.g., genome editing reagents and other biomolecules. The VLPs and mVLPs described herein can deliver a wide variety of cargo including but not limited to DNA only, DNA+RNA+protein, DNA+protein, RNA+protein, or protein only. mVLPs can control the form of the cargo (DNA, protein, and/or RNA).

### Phospholipid Bilayer Recruitment Domains

Conventional VLPs that have been engineered to encapsulate and deliver protein-based cargo commonly fuse cargo to the INT or GAG polyprotein.^{25-27,29,30,39,40} After transient transfection of production plasmid DNA constructs, these protein fusions are translated in the cytosol of conventional VLP production cell lines, the gag matrix is acetylated and recruited to the cell membrane, and the gag fusions are encapsulated (transient transfected DNA is also unintentionally and passively encapsulated) within VLPs as VLPs bud off of the membrane into extracellular space.

In contrast, in some embodiments, proteins can be packaged into the mVLPs by fusing select phospholipid bilayer recruitment domains, preferably human protein-derived phospholipid bilayer recruitment domains to protein-based cargo (e.g., as shown in Table 6).

One such human protein-derived phospholipid bilayer recruitment domain used for this purpose is a human pleckstrin homology (PH) domain. PH domains interact with phosphatidylinositol lipids and proteins within biological membranes, such as PIP2, PIP3, βγ-subunits of GPCRs, and PKC.^{41,42} Alternatively, the human Arc protein can be fused to protein-based cargo to recruit cargo to the cytosolic side of the phospholipid bilayer.⁴³ These human protein-derived phospholipid bilayer recruitment domains, or variants thereof (e.g., as shown in Table 6) can be fused to the N-terminus or C-terminus of protein-based cargo via polypeptide linkers of variable length regardless of the location or locations of one or more nuclear localization sequence(s) (NLS) within the cargo. Preferably, the linker between protein-based cargo and the phospholipid bilayer recruitment domain is a polypeptide linker 5-20, e.g., 8-12, e.g., 10, amino acids in length primarily composed of glycines and serines. The human protein-derived phospholipid bilayer recruitment domain localizes the cargo to the cytosolic face of the phospholipid bilayer and this protein cargo is packaged within mVLPs that utilize an envelope glycoprotein to trigger budding-off of particles from the producer cell into extracellular space. These human protein-derived domains and proteins can facilitate for localization of cargo to the cytosolic face of the plasma membrane within the mVLP production cells, and they also allow for cargo to localize to the nucleus of mVLP-transduced cells without the utilization of exogenous retroviral gag/pol or chemical and/or light-based dimerization systems. The delivery of Cas9, for example, is significantly more efficiently loaded as cargo into particles with fusion to a phospholipid bilayer recruitment domain compared to without fusion to a phospholipid bilayer recruitment domain.

### VLP/mVLP-Mediated Delivery of DNAs, Proteins and RNAs

The VLPs and mVLPs described herein (e.g., comprising tENV proteins) can package and deliver biomolecule cargo. “Cargo” refers to a any payload that can be delivered, including chemicals, e.g., small molecule compounds, and biomolecules, including DNA, RNA, peptide nucleic acid (PNA), RNP, proteins, and combinations thereof, including combinations of DNA and RNP, RNP, combinations of DNA and proteins, or proteins, as well as viruses and portions thereof, e.g., for therapeutic or diagnostic use, or for the applications of genome editing, epigenome modulating, and/or transcriptome modulation. RNA in this context includes, for example, single guide RNA (sgRNA), Clustered Regularly Interspaced Palindromic Repeat (CRISPR) RNA (crRNA), and/or mRNA coding for cargo. Other exemplary nucleic acids can include specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, crRNA, tracrRNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, or internal ribosomal entry site containing RNA). Combinations of the above cargos (e.g., AAV particles and/or ribonucleoprotein (RNP) complexes comprising RNA and protein, e.g., guide RNA/CRISPR Cas protein complexes) can also be included.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, the cargo is limited by the diameter of the particles, e.g., which in some embodiments can range from 30 nm to 500 nm.

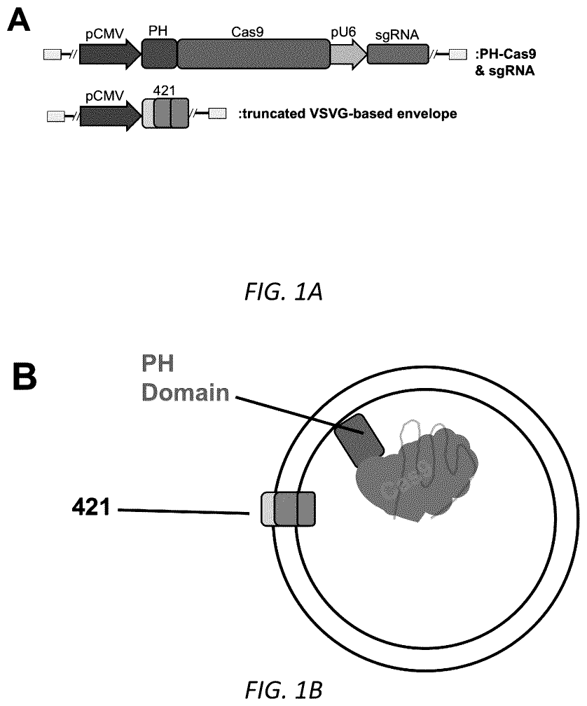

In some embodiments, the cargo can include a combination of DNA and RNA; for example, the VLPs or mVLPs can be produced via transient transfection of a production cell line. DNA that is transfected into cells will possess size-dependent mobility such that a fraction of the transfected DNA will remain in the cytosol while another fraction of the transfected DNA will localize to the nucleus.⁴⁴⁻⁴⁶ A fraction of the transfected DNA in the nucleus will be expressed components needed to create the VLPs/mVLPs and another fraction in the cytosol/near the plasma membrane will be encapsulated and delivered in VLPs/mVLPs. See, e.g., FIGS. 1-4 of WO 2022/020800.

Cargo developed for applications of genome or gene editing also includes CRISPR-Cas nucleases and fusions and variants thereof, e.g., prime editors, and base editors. Nucleases include ZFNs and Transcription activator-like effector nucleases (TALENs) that comprise a FokI or AcuI nuclease domain; and CRISPR Cas proteins or a functional derivative thereof (e.g., as shown in Table 2) (ZFNs are described, for example, in United States Patent Publications 20030232410; 20050208489; 20050026157; 20050064474; 20060188987; 20060063231; and International Publication WO 07/014275) (TALENs are described, for example, in United States Patent Publication U.S. Pat. No. 9,393,257B2; and International Publication WO2014134412A1) (CRISPR Cas proteins are described, for example, in United States Patent Publications U.S. Pat. No. 8,697,359B1; US20180208976A1; and International Publications WO2014093661A2; WO2017184786A8).³⁴⁻³⁶ Base editors can include any CRISPR based nuclease orthologs (wt, nickase, or catalytically inactive (CI)), e.g., as shown in Table 2, fused at the N-terminus to a nucleotide deaminase or nucleoside deaminase or a functional derivative thereof (e.g., as shown in Table 3), or comprising a deaminase domain inlaid internally, with or without a fusion at the C-terminus to one or multiple uracil glycosylase inhibitors (UGIs) using polypeptide linkers of variable length (Base editors are described, for example, in United States Patent Publications US20150166982A1; US20180312825A1; U.S. Ser. No. 10/113,163B2; and International Publications WO2015089406A1; WO2018218188A2; WO2017070632A2; WO2018027078A8; WO2018165629A1).³⁷,38 In addition, prime editors are also compatible with mVLP delivery modalities (Prime editors are described, for example, in Anzalone et al., Nature. 2019 December; 576(7785):149-157). Prime editors can be delivered, e.g., as fusions of Cas nickase to a reverse transcriptase or as separate components (see, e.g., Grunewald et al., Nat Biotechnol. 2022 Sep. 26. doi: 10.1038/s41587-022-01473-1; and Liu et al., Nat Biotechnol. 2022 September;40(9):1388-1393).

Cargo designed for the purposes of epigenome modulating includes CRISPR Cas proteins, zinc fingers (ZFs) and TALEs fused to an epigenome/epigenetic modulating agent or combination of epigenome/epigenetic modulating agent or a functional derivative thereof connected together by one or more variable length polypeptide linkers. Exemplary epigenetic modulating agents include CRISPR-Cas proteins (e.g., nickases or catalytically inactive Cas) fused to DNA methylases, histone acetyltransferases, and deacetylases, as well as transcriptional activators or repressors (see, e.g., Tables 2 & 4). Examples include, e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HPlu or HP10; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) In some embodiments, the sequence of the cargo is at least 95% identical to a sequence set forth herein.

sgRNAs can complex with genome editing reagents during the packaging process to be co-delivered within VLPs/mVLPs as described herein. Also, linear or circular RNAs encoding cargo or edits that are to be installed by a prime editor could be co-packaged with genome editing reagents that are fused to RNA binding proteins, such as MS2, PP7, COM, or TAR hairpin binding protein (TBP) or human SLBP. Cargo designed for the purposes of transcriptome editing includes CRISPR Cas proteins or any functional derivatives thereof (e.g., as shown in Table 5) or CRISPR Cas proteins or any functional derivatives thereof (e.g., as shown in Table 5) fused to nucleotide deaminases or nucleoside deaminases (e.g., as shown in Table 3) by one or more variable length polypeptide linkers.

The cargo can also include any therapeutically or diagnostically useful protein, DNA, RNP, or combination of DNA, protein and/or RNP. See, e.g., WO2014005219; U.S. Ser. No. 10/137,206; US20180339166; U.S. Pat. No. 5,892,020A; EP2134841B1; WO2007020965A1. For example, cargo encoding or composed of nuclease or base editor proteins or RNPs or derivatives thereof can be delivered to retinal cells for the purposes of correcting a splice site defect responsible for Leber Congenital Amaurosis type 10. In the mammalian inner ear, VLP/mVLP delivery of base editing reagents or HDR promoting cargo to sensory cells such as cochlear supporting cells and hair cells for the purposes of editing β-catenin (β-catenin Ser 33 edited to Tyr, Pro, or Cys) in order to better stabilize β-catenin could help reverse hearing loss.

In another application, VLP/mVLP delivery of RNA editing reagents or proteome perturbing reagents could cause a transitory reduction in cellular levels of one or more specific proteins of interest (potentially at a systemic level, in a specific organ or a specific subset of cells, such as a tumor), and this could create a therapeutically actionable window when secondary drug(s) could be administered (this secondary drug is more effective in the absence of the protein of interest or in the presence of lower levels of the protein of interest). For example, mVLP delivery of RNA editing reagents or proteome perturbing reagents could trigger targeted degradation of MAPK and PI3K/AKT proteins and related mRNAs in vemurafenib/dabrafenib-resistant BRAF-driven tumor cells, and this could open a window for the administration of vemurafenib/dabrafenib because BRAF inhibitor resistance is temporarily abolished (resistance mechanisms based in the MAPK/PI3K/AKT pathways are temporarily downregulated by VLP/mVLP cargo). This example is especially pertinent when combined with VLP/mVLPs that are antigen inducible and therefore specific for tumor cells. Alternatively, the transitory reduction in cellular levels of a specific protein of interest may itself have therapeutic benefit.

In some embodiments, VLPs and mVLPs described herein could be used deliver factors, e.g., including the Yamanaka factors Oct3/4, Sox2, Klf4, and c-Myc, to cells such as human or mouse fibroblasts, in order to generate induced pluripotent stem cells or to deliver factors that induce forward differentiation or trans-differentiation into a specific cell-type.

In some embodiments, VLPs and mVLPs described herein could deliver dominant-negative forms of proteins in order to elicit a therapeutic effect.

VLPs and mVLPs described herein that are antigen-specific (e.g., tumor-antigen specific) could be targeted to cancer cells in order to deliver proapoptotic proteins BIM, BID, PUMA, NOXA, BAD, BIK, BAX, BAK and/or HRK in order to trigger apoptosis of cancer cells. Tumor antigens are known in the art.

90% of pancreatic cancer patients present with unresectable disease. Around 30% of patients with unresectable pancreatic tumors will die from local disease progression, so it is desirable to treat locally advanced pancreatic tumors with ablative radiation, but the intestinal tract cannot tolerate high doses of radiation needed to cause tumor ablation. Selective radioprotection of the intestinal tract enables ablative radiation therapy of pancreatic tumors while minimizing damage done to the surrounding gastrointestinal tract. To this end, VLPs and mVLPs described herein could be loaded with dCas9 fused to the transcriptional repressor KRAB and guide RNA targeting EGLN. EGLN inhibition has been shown to significantly reduce gastrointestinal toxicity from ablative radiation treatments because it causes selective radioprotection of the gastrointestinal tract but not the pancreatic tumor.⁴⁷ Such fusion proteins, VLPs and mVLPs described herein, and methods of making and using the same are provided herein.

Unbound steroid receptors reside in the cytosol. After binding to ligands, these receptors will translocate to the nucleus and initiate transcription of response genes. VLPs and mVLPs described herein could deliver single chain variable fragment (scFv) antibodies to the cytosol of cells that bind to and disrupt cytosolic steroid receptors. For example, the scFv could bind to the glucocorticoid receptor and prevent it from binding dexamethasone, and this would prevent transcription of response genes, such as metallothionein 1E that has been linked to tumorigenesis.⁴⁸ VLPs and mVLPs described herein can be indicated for treatments that involve targeted disruption of proteins. For example, VLPs and mVLPs described herein can be utilized for targeting and disrupting proteins in the cytosol of cells by delivering antibodies/scFvs to the cytosol of cells. Classically, delivery of antibodies through the plasma membrane to the cytosol of cells has been notoriously difficult and inefficient. This mode of protein inhibition is similar to how a targeted small molecule binds to and disrupts proteins in the cytosol and could be useful for the treatment of a diverse array of diseases.⁴⁹⁻⁵¹ Such fusion proteins, VLPs and mVLPs described herein, and methods of making and using the same are included herein.

In addition, the targeting of targeted small molecules is limited to proteins of a certain size that contain binding pockets which are relevant to catalytic function or protein-protein interactions. scFvs are not hampered by these limitations because scFvs can be generated that bind to many different moieties of a protein in order to disrupt catalysis and interactions with other proteins. For example, RAS oncoproteins are implicated across a multitude of cancer subtypes, and RAS is one of the most frequently observed oncogenes in cancer. For instance, the International Cancer Genome Consortium found KRAS to be mutated in 95% of their Pancreatic Adenocarcinoma samples. RAS isoforms are known to activate a variety of pathways that are dysregulated in human cancers, like the PI3K and MAPK pathways. Despite the aberrant roles RAS plays in cancer, no efficacious pharmacologic direct or indirect small molecule inhibitors of RAS have been developed and approved for clinical use. One strategy for targeting RAS could be VLPs and mVLPs described herein that can deliver specifically to cancer cells scFvs that bind to and disrupt the function of multiple RAS isoforms.⁴⁹⁻⁵¹

### VLP/mVLP Composition, Production, Purification and Applications

The VLPs and mVLPs described herein (e.g., comprising tENV proteins) can be produced from producer cell lines that are either transiently transfected with at least one plasmid and/or that stably express constructs that have been integrated into the producer cell line genomic DNA. This, in some embodiments, the VLPs and mVLPs described herein can be produced and package protein-based cargo by integrating all production DNA constructs into the genomic DNA of production cell lines. Once cell lines are created, protein delivery VLPs and mVLPs can be produced in a constitutive or inducible fashion.

Some or all of the components for producing VLPs or mVPLs can be transiently expressed. In some embodiments, if a single plasmid is used in the transfection, it should comprise sequences encoding tENV as described herein, cargo (e.g., a therapeutic protein or a gene editing reagent such as a zinc finger, transcription activator-like effector (TALE), and/or CRISPR-based genome editing/modulating protein and/or RNP having a sequence such as those found in Tables 2, 3, 4 & 5; in some embodiments, the sequence is at least 95% identical to a sequence set forth herein), with or without fusion to a phospholipid bilayer recruitment domain (e.g., as shown in Table 6), and a guide RNA, if necessary. Preferably, two to three plasmids are used in the transfection. These two to three plasmids can include the following (any two or more can be combined in a single plasmid):
1. A plasmid comprising sequences encoding cargo, e.g., a therapeutic protein or a genome editing reagent, with or without a fusion to a phospholipid bilayer recruitment domain.
2. A plasmid comprising sequences encoding tENV as described herein.
3. If the genome editing reagent from plasmid 1 requires one or more guide RNAs, a plasmid comprising one or more guide RNAs apposite for the genome editing reagent.
   If it is desired to deliver a type of DNA molecule other than plasmid(s), the above-mentioned transfection can be performed with double-stranded closed-end linear DNA, episome, mini circle, double-stranded oligonucleotide and/or other specialty/modified DNA, RNA, AAV, adenovirus, anellovirus, or peptide nucleic acid (PNA) molecules. Alternatively, the producer cell line can be made to stably express any one or more of the constructs (1 through 3) described in the transfection above.

In some embodiments, the methods can include using cells that have or have not been manipulated to express any exogenous proteins except for a tENV viral envelope protein (as described herein, e.g., as shown in Table 1), and, if desired, a phospholipid bilayer recruitment domain (e.g., as shown in Table 6); in other words, no cargo is expressed. In this embodiment, the “empty” particles that are produced can be loaded with cargo and/or small molecules by utilizing incubation, nucleofection, lipid, polymer, or CaCl₂) transfection, sonication, freeze thaw, and/or heat shock of purified particles mixed with cargo. In some embodiments, producer cells do not express any gag, pro, or pol protein. This type of loading allows for cargo to be unmodified by fusions to phospholipid bilayer recruitment domains and represents a significant advancement from previous VLP technology.

The plasmids, or other types of specialty DNA molecules known in the art or described herein, can also preferably include other elements to drive expression or translation of the encoded sequences, e.g., a promoter sequence; an enhancer sequence, e.g., 5′ untranslated region (UTR) or a 3′ UTR; a polyadenylation site; an insulator sequence; or another sequence that increases or controls expression (e.g., an inducible promoter element).

Appropriate producer cell lines can include primary or stable human cell lines refractory to the effects of transfection reagents and fusogenic effects due to virally-derived glycoproteins. Examples of appropriate cell lines include Human Embryonic Kidney (HEK) 293 cells, HEK293 T/17 SF cells kidney-derived Phoenix-AMPHO cells, and placenta-derived BeWo cells. For example, such cells could be selected for their ability to grow as adherent cells, or suspension cells. In some embodiments, the producer cells can be cultured in classical DMEM under serum conditions, serum-free conditions, or exosome-free serum conditions. mVLPs can be produced from cells that have been derived from patients (autologous mVLPs) and other FDA-approved cell lines (allogenic mVLPs) as long as these cells can be transfected with DNA constructs that encode the aforementioned mVLP production components by various techniques known in the art.

In addition, if it is desirable, more than one genome editing reagent can be included in the transfection. The DNA constructs can be designed to overexpress proteins in the producer cell lines. The plasmid backbones, for example, used in the transfection can be familiar to those skilled in the art, such as the pCDNA3 backbone that employs the CMV promoter for RNA polymerase II transcripts or the U6 promoter for RNA polymerase III transcripts. Various techniques known in the art may be employed for introducing nucleic acid molecules into producer cells. Such techniques include chemical-facilitated transfection using compounds such as calcium phosphate, cationic lipids, cationic polymers, liposome-mediated transfection, such as cationic liposome like LIPOFECTAMINE (LIPOFECTAMINE 2000 or 3000 and TransIT-X2), polyethyleneimine, non-chemical methods such as electroporation, particle bombardment, or microinjection.

A human producer cell line that stably expresses the necessary VLP or mVLP components in a constitutive and/or inducible fashion can be used for production of VLPs or mVLPs. For example, mVLPs can be produced from cells that have been derived from patients (autologous mVLPs) and other FDA-approved cell lines (allogenic mVLPs) if these cells have been converted into stable cell lines that express the aforementioned mVLP components.

Also provided herein are the producer cells themselves.

### Production of Cargo-Loaded VLPs/mVLPs and Compositions

Preferably the VLPs/mVLPs described herein are harvested from cell culture medium supernatant 36-48 hours post-transfection, or when the VLPs/mVLPs are at the maximum concentration in the medium of the producer cells (the producer cells are expelling particles into the media and at some point in time, the particle concentration in the media will be optimal for harvesting the particles). Supernatant can be purified by any known methods in the art, such as centrifugation, ultracentrifugation, precipitation, ultrafiltration, and/or chromatography. In some embodiments, the supernatant is first filtered, e.g., to remove particles larger than 1 μm, e.g., through 0.45 pore size polyvinylidene fluoride hydrophilic membrane (Millipore Millex-HV) or 0.8 m pore size mixed cellulose esters hydrophilic membrane (Millipore Millex-AA). After filtration, the supernatant can be further purified and concentrated, e.g., using ultracentrifugation, e.g., at a speed of 80,000 to 100,000×g at a temperature between 1° C. and 5° C. for 1 to 2 hours, or at a speed of 8,000 to 15,000 g at a temperature between 1° C. and 5° C. for 10 to 16 hours. After this centrifugation step, the VLPs/mVLPs are concentrated in the form of a centrifugate (pellet), which can be resuspended to a desired concentration, mixed with transduction-enhancing reagents, subjected to a buffer exchange, or used as is. In some embodiments, VLP/mVLP-containing supernatant can be filtered, precipitated, centrifuged and resuspended to a concentrated solution. For example, polyethylene glycol (PEG), e.g., PEG 8000, or antibody-bead conjugates that bind to VLP/mVLP-surface proteins or membrane components can be used to precipitate particles. Purified particles are stable and can be stored at 4° C. for up to a week or −80° C. for years without losing appreciable activity.

Preferably, VLPs/mVLPs are resuspended or undergo buffer exchange so that particles are suspended in an appropriate carrier. In some embodiments, buffer exchange can be performed by ultrafiltration (Sartorius Vivaspin 500 MWCO 100,000). An exemplary appropriate carrier for VLPs/mVLPs to be used for in vitro applications would preferably be a cell culture medium that is suitable for the cells that are to be transduced by VLPs/mVLPs. Transduction-enhancing reagents that can be mixed into the purified and concentrated VLPs/mVLPs solution for in vitro applications include reagents known by those familiar with the art (Miltenyi Biotec Vectofusin-1, Millipore Polybrene, Takara Retronectin, Sigma Protamine Sulfate, and the like). After VLPs/mVLPs in an appropriate carrier are applied to the cells to be transduced, transduction efficiency can be further increased by centrifugation. Preferably, the plate containing VLPs/mVLPs applied to cells can be centrifuged at a speed of 1,150 g at room temperature for 30 minutes. After centrifugation, cells are returned into the appropriate cell culture incubator (humidified incubator at 37° C. with 5% CO₂).

An appropriate carrier for VLPs/mVLPs to be administered to a mammal, especially a human, would preferably be a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and and similar solutions or mixtures of such salts), or dry, especially freeze-dried compositions which upon addition, depending on the case, of sterilized water or physiological saline, permit the constitution of injectable solutions. Another appropriate pharmaceutical form would be aerosolized particles for administration by intranasal inhalation or intratracheal intubation.

The pharmaceutical forms suitable for injectable use include sterile aqueous solutions or suspensions. The solution or suspension may comprise additives which are compatible with VLPs/mVLPs and do not prevent VLPs/mVLPs entry into target cells. In all cases, the form must be sterile and must be fluid to the extent that the form can be administered with a syringe. It must be stable under the conditions of manufacture and storage and must be preserved against the contaminating action of microorganisms, such as bacteria and fungi. An example of an appropriate solution is a buffer, such as phosphate buffered saline.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Compositions comprising cargo-loaded VLPs/mVLPs as described herein can be included in a container, pack, or dispenser together with instructions for administration.

TABLE 2
Exemplary Potential Cas9 and Cas12a orthologs

| DNA-binding Cas | Enzyme class | Nickase mutation | CI mutations |
|---|---|---|---|
| ortholog | | | |
| SpCas9 | Type II-A | D10A | D10A, H840A |
| SaCas9 | Type II-A | D10A | D10A, |
| CjCas9 | Type II-C | D8A | D8A, |
| NmeCas9 | Type II-C | D16A | D16A, H588A |
| AsCas12a | Type II-C | | D908A, E993A |
| LbCas12a | Type II-C | | D832A, E925A |

Nickase mutation residues represents a position of the enzyme either known to be required for catalytic activity of the conserved RuvC nuclease domain or predicted to be required for this catalytic activity based on sequence alignment to CjCas9 where structural information is lacking (* indicates which proteins lack sufficient structural information). All positional information refers to the wild-type protein sequences acquired from uniprot.org.

TABLE 3
Exemplary Deaminase domains and their
substrate sequence preferences.

| Deaminase | Nucleotide sequence preference |
|---|---|
| hAID | 5′-WRC |
| rAPOBEC1* | 5′-TC ≥ CC ≥ AC > GC |
| mAPOBEC3 | 5′-TYC |
| hAPOBEC3A | 5′-TCG |
| hAPOBEC3B | 5′-TCR > TCT |
| hAPOBEC3C | 5′-WYC |
| hAPOBEC3F | 5′-TTC |
| hAPOBEC3G | 5′-CCC |
| hAPOBEC3H | 5′-TTCA ~ TTCT ~ TTCG > ACCCA > TGCA |
| E. coli TadA | A |
| hAdar1 | A |
| hAdar2 | A |

Nucleotide positions that are poorly specified or are permissive of two or more nucleotides are annotated according to IUPAC codes, where W = A or T, R = A or G, and Y = C or T. “h” before the deaminase name indicates Homo sapiens origin. “m” before the deaminase name indicates Mus musculus origin. “r” before the deaminase name indicates Rattus origin.

TABLE 4
Exemplary Epigenetic modulating domains.

| Epigenetic modulator | Epigenetic modulation |
|---|---|
| VP16 | transcriptional activation |
| VP64 | transcriptional activation |
| P65 | transcriptional activation |
| RTA | transcriptional activation |
| KRAB | transcriptional repression |
| MeCP2 | transcriptional repression |
| TET1 | Methylation |
| DNMT3A | Methylation |

TABLE 5
Exemplary CRISPR based RNA-guided RNA binding enzymes

| RNA-binding Cas ortholog | Enzyme class |
|---|---|
| LshCas13a | Type-VI |
| LwaCas13a | Type-VI |

TABLE 6
Exemplary Plasma membrane recruitment domains

| Plasma membrane recruitment domain | Substitution(s) |
|---|---|
| Pleckstrin homology domain of human | |
| phospholipase Cδ1 (hPLCδ1) | |
| Pleckstrin homology domain of human | R40L⁵² |
| phospholipase Cδ1 (hPLCδ1) | |
| Pleckstrin homology domain of human | |
| AKT1 (hAKT1) | |
| Mutant Pleckstrin homology domain of | E17K⁵³ |
| human AKT1 | |
| Pleckstrin homology domain of human 3- | |
| phosphoinositide-dependent protein | |
| kinase 1 (hPDPK1) | |
| Mutant Pleckstrin homology domain of | K14R⁵⁶ |
| human AKT1 | |
| Mutant Pleckstrin homology domain of | K8R⁵⁷ |
| human AKT1 | |
| Mutant Pleckstrin homology domain of | T72A⁵⁸ |
| human AKT1 | |
| Mutant Pleckstrin homology domain of | T92A⁵⁹ |
| human AKT1 | |
| Mutant Pleckstrin homology domain of | R25C⁵² |
| human AKT1 | |
| Mutant Pleckstrin homology domain of | T34D⁵⁴ |
| human AKT1 | |
| Mutant Pleckstrin homology domain of | T34F⁵⁴ |
| human AKT1 | |
| Mutant Pleckstrin homology domain of | T34L⁵⁴ |
| human AKT1 | |
| Mutant Pleckstrin homology domain of | T81Y⁵⁵ |
| human AKT1 | |
| Mutant Pleckstrin homology domain of | K142A, H143A, |
| human AKT1 | R144A⁶⁰ |
| Mutant Pleckstrin homology domain of | T101C⁶¹ |
| human AKT1 | |
| Pleckstrin homology domain of Human | |
| Dapp1 | |
| Pleckstrin homology domain of Human | |
| GRP1 | |
| Pleckstrin homology domain of Human | R284C⁵² |
| GRP1 | |
| Pleckstrin homology domain of Human | |
| OSBP1 | |
| Pleckstrin homology domain of Human | R108E⁵² |
| OSBP1 | |
| Pleckstrin homology domain of Human | |
| ARNO (CYTH2) | |
| Pleckstrin homology domain of Human | R279C⁵² |
| ARNO (CYTH2) | |
| Pleckstrin homology domain of Human | |
| BTK1 | |
| Pleckstrin homology domain of Human | R28C⁵² |
| BTK1 | |
| FYVE domain of Human EEA1 | |
| FYVE domain of Human EEA1 | R1375L⁵² |
| PX domain of p40^{phox} (NCF4) | |
| PX domain of p40^{phox} (NCF4) | R58L⁵² |
| Pleckstrin homology domain of Human | |
| FAPP1 | |
| Pleckstrin homology domain of Human | |
| CERT | |
| Pleckstrin homology domain of Human | |
| PHLPP1 | |
| Pleckstrin homology domain of Human | |
| SWAP70 | |
| Pleckstrin homology domain of Human | R223E and |
| SWAP70 | R224E⁶² |
| Pleckstrin Homology Domain of Human | |
| PKD | |
| Pleckstrin homology domain of Human | |
| MAPKAP1 | |
| Pleckstrin homology domain of Human | |
| Son Of Sevenless Homolog 2 | |
| Pleckstrin homology domain of Human | |
| Dynamin | |
| Pleckstrin homology domain of Human | |
| BCR | |
| Pleckstrin homology domain of Human | |
| DBS | |

### Exemplary Sequences

In some embodiments, the sequence of a protein or nucleic acid used in a composition or method described herein is at least 80%, 85%, 90%, 95%, 97%, 98%, or 99% identical to a sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Prime Editor: spCas9 H840A-MMLV Reverse
Transcriptase (delta RNase H domain):
(SEQ ID NO: 90)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPWVALNPA
TLLPLPEEGLQHNCLSGGSKRTADGSEPKKKRKVGS
Rattus norvegicus & synthetic: APOBEC1-XTEN
L8-nspCas9-UGI-SV40 NLS
(SEQ ID NO: 91)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKE
DIQKAQVSGQGDSLHEHIANLAGSPAIKKGILQTVKWDELVKVMGRHKPE
NIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVIT
LKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQL
FVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYE
TRIDLSQLGGDSGGSTNLSDIIEKETGKQLVIQESILMLPEEVEEVIGNK
PESDILVHTAYDESTDENVMLLTSDAPEYKPWALVIQDSNGENKIKMLSG
GSPKKKRKV
Homo sapiens: AID
(SEQ ID NO: 92)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
Homo sapiens: AIDv solubility variant
lacking N-terminal RNA-binding region
(SEQ ID NO: 93)
LMDPHIFTSNFNNGIGRHKTYLCYEVERLDSATSFSLDFGYLRNKNGCHV
ELLFLRYISDWDLDPGRCYRVTWFTSWSPCYDCARHVADFLRGNPNLSLR
IFTARLYFCEDRKAEPEGLRRLHRAGVQIAIMTFKDYFYCWNTFVENHER
TFKAWEGLHENSVRLSRQLRRILLPLYEVDDLRDAFRTLGL
Homo sapiens: AIDv solubility variant
lacking N-terminal RNA-binding region and
the C-terminal poorly structured region
(SEQ ID NO: 94)
MDPHIFTSNFNNGIGRHKTYLCYEVERLDSATSFSLDFGYLRNKNGCHVE
LLFLRYISDWDLDPGRCYRVTWFTSWSPCYDCARHVADFLRGNPNLSLRI
FTARLYFCEDRKAEPEGLRRLHRAGVQIAIMTFKDYFYCWNTFVENHERT
FKAWEGLHENSVRLSRQLRRILLPL
Rattus norvegicus: APOBEC1
(SEQ ID NO: 95)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK
Mus musculus: APOBEC3
(SEQ ID NO: 96)
MGPFCLGCSHRKCYSPIRNLISQETFKFHFKNLGYAKGRKDTFLCYEVTR
KDCDSPVSLHHGVFKNKDNIHAEICFLYWFHDKVLKVLSPREEFKITWYM
SWSPCFECAEQIVRFLATHHNLSLDIFSSRLYNVQDPETQQNLCRLVQEG
AQVAAMDLYEFKKCWKKFVDNGGRRFRPWKRLLTNFRYQDSKLQEILRRM
DPLSEEEFYSQFYNQRVKHLCYYHRMKPYLCYQLEQFNGQAPLKGCLLSE
KGKQHAEILFLDKIRSMELSQVTITCYLTWSPCPNCAWQLAAFKRDRPDL
ILHIYTSRLYFHWKRPFQKGLCSLWQSGILVDVMDLPQFTDCWTNFVNPK
RPFRPWKGLEIISRRTQRRLRRIKESWGLQDLVNDFGNLQLGPPMSN
Mus musculus: APOBEC3 catalytic domain
(SEQ ID NO: 97)
MGPFCLGCSHRKCYSPIRNLISQETFKFHFKNLGYAKGRKDTFLCYEVTR
KDCDSPVSLHHGVFKNKDNIHAEICFLYWFHDKVLKVLSPREEFKITWYM
SWSPCFECAEQIVRFLATHHNLSLDIFSSRLYNVQDPETQQNLCRLVQEG
AQVAAMDLYEFKKCWKKFVDNGGRRFRPWKRLLTNFRYQDSKLQEILRR
Homo sapiens: APOBEC3A
(SEQ ID NO: 98)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN
Homo sapiens: APOBEC3G
(SEQ ID NO: 99)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYDDQGRCQEGLRTLAEAGAKISIMTYSEFKHCWDTF
VDHQGCPFQPWDGLDEHSQDLSGRLRAILQNQEN
Homo sapiens: APOBEC3G catalytic domain
(SEQ ID NO: 100)
PPTFTFNFNNEPWVRGRHETYLCYEVERMHNDTWVLLNQRRGFLCNQAPH
KHGFLEGRHAELCFLDVIPFWKLDLDQDYRVTCFTSWSPCFSCAQEMAKF
ISKNKHVSLCIFTARIYDDQGRCQEGLRTLAEAGAKISIMTYSEFKHCWD
TFVDHQGCPFQPWDGLDEHSQDLSGRLRAILQNQEN
Homo sapiens: APOBEC3H
(SEQ ID NO: 101)
MALLTAETFRLQFNNKRRLRRPYYPRKALLCYQLTPQNGSTPTRGYFENK
KKCHAEICFINEIKSMGLDETQCYQVTCYLTWSPCSSCAWELVDFIKAHD
HLNLGIFASRLYYHWCKPQQKGLRLLCGSQVPVEVMGFPKFADCWENFVD
HEKPLSFNPYKMLEELDKNSRAIKRRLERIKIPGVRAQGRYMDILCDAEV
Homo sapiens: APOBEC3F
(SEQ ID NO: 102)
MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCV
AKLAEFLAEHPNVTLTISAARLYYYWERDYRRALCRLSQAGARVKIMDDE
EFAYCWENFVYSEGQPFMPWYKFDDNYAFLHRTLKEILRNPMEAMYPHIF
YFHFKNLRKAYGRNESWLCFTMEWKHHSPVSWKRGVFRNQVDPETHCHAE
RCFLSWFCDDILSPNTNYEVTWYTSWSPCPECAGEVAEFLARHSNVNLTI
FTARLYYFWDTDYQEGLRSLSQEGASVEIMGYKDFKYCWENFVYNDDEPF
KPWKGLKYNFLFLDSKLQEILE
Homo sapiens: APOBEC3F catalytic domain
(SEQ ID NO: 103)
KEILRNPMEAMYPHIFYFHFKNLRKAYGRNESWLCFTMEVVKHHSPVSWK
RGVFRNQVDPETHCHAERCFLSWFCDDILSPNTNYEVTWYTSWSPCPECA
GEVAEFLARHSNVNLTIFTARLYYFWDTDYQEGLRSLSQEGASVEIMGYK
DFKYCWENFVYNDDEPFKPWKGLKYNFLFLDSKLQEILE
Escherichia coli: TadA
(SEQ ID NO: 104)
MKRTADGSEFESPKKKRKVSEVEFSHEYWMRHALTLAKRAWDEREVPVGA
VLVHNNRVIGEGWNRPIGRHDPTAHAEIMALRQGGLVMQNYRLIDATLYV
TLEPCVMCAGAMIHSRIGRVVFGARDAKTGAAGSLMDVLHHPGMNHRVEI
TEGILADECAALLSDFFRMRRQEIKAQKKAQSSTDSGGSSGGSSGSETPG
TSESATPESSGGSSGGSSEVEFSHEYWMRHALTLAKRARDEREVPVGAVL
VLNNRVIGEGWNRAIGLHDPTAHAEIMALRQGGLVMQNYRLIDATLYVTF
EPCVMCAGAMIHSRIGRVVFGVRNAKTGAAGSLMDVLHYPGMNHRVEITE
GILADECAALLCYFFRMPRQVFNAQKKAQSSTD
Homo sapiens: Adar1
(SEQ ID NO: 105)
MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLG
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISE
SLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAE
RMGFTEVTPVTGASLRRTMLLLSRSPEAQPKTLPLTGSTFHDQIAMLSHR
CFNTLTNSFQPSLLGRKILAAIIMKKDSEDMGVVVSLGTGNRCVKGDSLS
LKGETVNDCHAEIISRRGFIRFLYSELMKYNSQTAKDSIFEPAKGGEKLQ
IKKTVSFHLYISTAPCGDGALFDKSCSDRAMESTESRHYPVFENPKQGKL
RTKVENGEGTIPVESSDIVPTWDGIRLGERLRTMSCSDKILRWNVLGLQG
ALLTHFLQPIYLKSVTLGYLFSQGHLTRAICCRVTRDGSAFEDGLRHPFI
VNHPKVGRVSIYDSKRQSGKTKETSVNWCLADGYDLEILDGTRGTVDGPR
NELSRVSKKNIFLLFKKLCSFRYRRDLLRLSYGEAKKAARDYETAKNYFK
KGLKDMGYGNWISKPQEEKNFYLCPV
Homo sapiens: Adar2
(SEQ ID NO: 106)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFNLH
LDQTPSRQPIPSEGLQLHLPQVLADAVSRLVLGKFGDLTDNFSSPHARRK
VLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSP
CGDARIFSPHEPILEEPADRHPNRKARGQLRTKIESGQGTIPVRSNASIQ
TWDGVLQGERLLTMSCSDKIARWNVVGIQGSLLSIFVEPIYFSSIILGSL
YHGDHLSRAMYQRISNIEDLPPLYTLNKPLLSGISNAEARQPGKAPNFSV
NWTVGDSAIEVINATTGKDELGRASRLCKHALYCRWMRVHGKVPSHLLRS
KITKPNVYHESKLAAKEYQAAKARLFTAFIKAGLGAWVEKPTEQDQFSLT
P
Streptococcus pyogenes: Cas9 Bipartite NLS
(SEQ ID NO: 107)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDGSGGGGSGKRTADGSEFEPKKKRKVSSGGDYK
DHDGDYKDHDIDYKDDDDK
Staphylococcus aureus: Cas9
(SEQ ID NO: 108)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG
Campylobacter jejuni: Cas9
(SEQ ID NO: 109)
MARILAFDIGISSIGWAFSENDELKDCGVRIFTKVENPKTGESLALPRRL
ARSARKRLARRKARLNHLKHLIANEFKLNYEDYQSFDESLAKAYKGSLIS
PYELRFRALNELLSKQDFARVILHIAKRRGYDDIKNSDDKEKGAILKAIK
QNEEKLANYQSVGEYLYKEYFQKFKENSKEFTNVRNKKESYERCIAQSFL
KDELKLIFKKQREFGFSFSKKFEEEVLSVAFYKRALKDFSHLVGNCSFFT
DEKRAPKNSPLAFMFVALTRIINLLNNLKNTEGILYTKDDLNALLNEVLK
NGTLTYKQTKKLLGLSDDYEFKGEKGTYFIEFKKYKEFIKALGEHNLSQD
DLNEIAKDITLIKDEIKLKKALAKYDLNQNQIDSLSKLEFKDHLNISFKA
LKLVTPLMLEGKKYDEACNELNLKVAINEDKKDFLPAFNETYYKDEVTNP
VVLRAIKEYRKVLNALLKKYGKVHKINIELAREVGKNHSQRAKIEKEQNE
NYKAKKDAELECEKLGLKINSKNILKLRLFKEQKEFCAYSGEKIKISDLQ
DEKMLEIDHIYPYSRSFDDSYMNKVLVFTKQNQEKLNQTPFEAFGNDSAK
WQKIEVLAKNLPTKKQKRILDKNYKDKEQKNFKDRNLNDTRYIARLVLNY
TKDYLDFLPLSDDENTKLNDTQKGSKVHVEAKSGMLTSALRHTWGFSAKD
RNNHLHHAIDAVIIAYANNSIVKAFSDFKKEQESNSAELYAKKISELDYK
NKRKFFEPFSGFRQKVLDKIDEIFVSKPERKKPSGALHEETFRKEEEFYQ
SYGGKEGVLKALELGKIRKVNGKIVKNGDMFRVDIFKHKKTNKFYAVPIY
TMDFALKVLPNKAVARSKKGEIKDWILMDENYEFCFSLYKDSLILIQTKD
MQEPEFVYYNAFTSSTVSLIVSKHDNKFETLSKNQKILFKNANEKEVIAK
SIGIQNLKVFEKYIVSALGEVTKAEFRQREDFKK
Neisseria meningitidis: Cas9
(SEQ ID NO: 110)
MAAFKPNSINYILGLDIGIASVGWAMVEIDEEENPIRLIDLGVRVFERAE
VPKTGDSLAMARRLARSVRRLTRRRAHRLLRTRRLLKREGVLQAANFDEN
GLIKSLPNTPWQLRAAALDRKLTPLEWSAVLLHLIKHRGYLSQRKNEGET
ADKELGALLKGVAGNAHALQTGDFRTPAELALNKFEKESGHIRNQRSDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAWVACSTVAMQQKITRFVRYKEMN
AFDGKTIDKETGEVLHQKTHFPQPWEFFAQEVMIRVFGKPDGKPEFEEAD
TLEKLRTLLAEKLSSRPEAVHEYVTPLFVSRAPNRKMSGQGHMETVKSAK
RLDEGVSVLRVPLTQLKLKDLEKMVNREREPKLYEALKARLEAHKDDPAK
AFAEPFYKYDKAGNRTQQVKAVRVEQVQKTGVWVRNHNGIADNATMVRVD
VFEKGDKYYLVPIYSWQVAKGILPDRAVVQGKDEEDWQLIDDSFNFKFSL
HPNDLVEVITKKARMFGYFASCHRGTGNINIRIHDLDHKIGKNGILEGIG
VKTALSFQKYQIDELGKEIRPCRLKKRPPVR
Acidaminococcus sp. Cas12a
(SEQ ID NO: 111)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSWVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN
Lachnospiraceae bacterium Cas12a:
(SEQ ID NO: 112)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQWVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH
Leptotrichia shahii Cas13a
(SEQ ID NO: 113)
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL
Leptotrichia wadei Cas13a
(SEQ ID NO: 114)
MKVTKVDGISHKKYIEEGKLVKSTSEENRTSERLSELLSIRLDIYIKNPD
NASEEENRIRRENLKKFFSNKVLHLKDSVLYLKNRKEKNAVQDKNYSEED
ISEYDLKNKNSFSVLKKILLNEDVNSEELEIFRKDVEAKLNKINSLKYSF
EENKANYQKINENNVEKVGGKSKRNIIYDYYRESAKRNDYINNVQEAFDK
LYKKEDIEKLFFLIENSKKHEKYKIREYYHKIIGRKNDKENFAKIIYEEI
QNVNNIKELIEKIPDMSELKKSQVFYKYYLDKEELNDKNIKYAFCHFVEI
EMSQLLKNYVYKRLSNISNDKIKRIFEYQNLKKLIENKLLNKLDTYVRNC
GKYNYYLQVGEIATSDFIARNRQNEAFLRNIIGVSSVAYFSLRNILETEN
ENGITGRMRGKTVKNNKGEEKYVSGEVDKIYNENKQNEVKENLKMFYSYD
FNMDNKNEIEDFFANIDEAISSIRHGIVHFNLELEGKDIFAFKNIAPSEI
SKKMFQNEINEKKLKLKIFKQLNSANVFNYYEKDVIIKYLKNTKFNFVNK
NIPFVPSFTKLYNKIEDLRNTLKFFWSVPKDKEEKDAQIYLLKNIYYGEF
LNKFVKNSKVFFKITNEVIKINKQRNQKTGHYKYQKFENIEKTVPVEYLA
IIQSREMINNQDKEEKNTYIDFIQQIFLKGFIDYLNKNNLKYIESNNNND
NNDIFSKIKIKKDNKEKYDKILKNYEKHNRNKEIPHEINEFVREIKLGKI
LKYTENLNMFYLILKLLNHKELTNLKGSLEKYQSANKEETFSDELELINL
LNLDNNRVTEDFELEANEIGKFLDFNENKIKDRKELKKFDTNKIYFDGEN
IIKHRAFYNIKKYGMLNLLEKIADKAKYKISLKELKEYSNKKNEIEKNYT
MQQNLHRKYARPKKDEKFNDEDYKEYEKAIGNIQKYTHLKNKVEFNELNL
LQGLLLKILHRLVGYTSIWERDLRFRLKGEFPENHYIEEIFNFDNSKNVK
YKSGQIVEKYINFYKELYKDNVEKRSIYSDKKVKKLKQEKKDLYIRNYIA
HFNYIPHAEISLLEVLENLRKLLSYDRKLKNAIMKSIVDILKEYGFVATF
KIGADKKIEIQTLESEKIVHLKNLKKKKLMTDRNSEELCELVKVMFEYKA
LE
Pleckstrin homology domain of Human ARNO:
(SEQ ID NO: 115)
NPDREGWLLKLGGGRVKTWKRRWFILTDNCLYYFEYTTDKEPRGIIPLEN
LSIREVDDPRKPNCFELYIPNNKGQLIKACKTEADGRVVEGNHMVYRISA
PTQEEKDEWIKSIQAAVS
Pleckstrin homology domain of Human ARNO
R279C:
(SEQ ID NO: 116)
NPDREGWLLKLGGGRVKTWKCRWFILTDNCLYYFEYTTDKEPRGIIPLEN
LSIREVDDPRKPNCFELYIPNNKGQLIKACKTEADGRVVEGNHMVYRISA
PTQEEKDEWIKSIQAAVS
FYVE domain of Human EEA1:
(SEQ ID NO: 117)
DNEVQNCMACGKGFSVTVRRHHCRQCGNIFCAECSAKNALTPSSKKPVRV
CDACFNDLQ
FYVE domain of Human EEA1 R1375L:
(SEQ ID NO: 118)
DNEVQNCMACGKGFSVTVRRHHCLQCGNIFCAECSAKNALTPSSKKPVRV
CDACFNDLQ
PX domain of p40^{phox} (NCF4):
(SEQ ID NO: 119)
DVAISANIADIEEKRGFTSHFVFVIEVKTKGGSKYLIYRRYRQFHALQSK
LEERFGPDSKSSALACTLPTLPAKVYVGVKQEIAEMRIPALNAYMKSLLS
LPVWVLMDEDVRIFFYQSPYDS
PX domain of p40^{phox} (NCF4) R58L:
(SEQ ID NO: 120)
DVAISANIADIEEKRGFTSHFVFVIEVKTKGGSKYLIYLRYRQFHALQSK
LEERFGPDSKSSALACTLPTLPAKVYVGVKQEIAEMRIPALNAYMKSLLS
LPVWVLMDEDVRIFFYQSPYDS
Pleckstrin homology domain of
Homo sapiens DAPP1
(SEQ ID NO: 121)
MQTGRTEDDLVPTAPSLGTKEGYLTKQGGLVKTWKTRWFTLHRNELKYFK
DQMSPEPIRILDLTECSAVQFDYSQERVNCFCLVFPFRTFYLCAKTGVEA
DEWIKILRWKLSQIRKQLNQGEGTIR
Pleckstrin homology domain of
Homo sapiens GRP1 (CYTH3)
(SEQ ID NO: 122)
PFKIPEDDGNDLTHTFFNPDREGWLLKLGGRVKTWKRRWFILTDNCLYYF
EYTTDKEPRGIIPLENLSIREVEDPRKPNCFELYNPSHKGQVIKACKTEA
DGRVVEGNHVVYRISAPSPEEKEEWMKSIKASISRDPFYDMLATRKRRIA
NKK
Pleckstrin homology domain of
Homo sapiens GRP1 (CYTH3) R284C:
(SEQ ID NO: 123)
MPFKIPEDDGNDLTHTFFNPDREGWLLKLGGRVKTWKCRWFILTDNCLYY
FEYTTDKEPRGIIPLENLSIREVEDPRKPNCFELYNPSHKGQVIKACKTE
ADGRWVEGNHVVYRISAPSPEEKEEWMKSIKASISRDPFYDMLATRKRRI
ANKK
Pleckstrin homology domain of Human OSBP1
(SEQ ID NO: 124)
MGSGSAREGWLFKWTNYIKGYQRRWFVLSNGLLSYYRSKAEMRHTCRGTI
NLATANITVEDSCNFIISNGGAQTYHLKASSEVERQRWVTALELAKAKAV
K
Pleckstrin homology domain of Human OSBP1
R108E:
(SEQ ID NO: 125)
MGSGSAREGWLFKWTNYIKGYQERWFVLSNGLLSYYRSKAEMRHTCRGTI
NLATANITVEDSCNFIISNGGAQTYHLKASSEVERQRWVTALELAKAKAV
K
Pleckstrin homology domain of Human BTK1
(SEQ ID NO: 126)
MAAVILESIFLKRSQQKKKTSPLNFKKRLFLLTVHKLSYYEYDFERGRRG
SKKGSIDVEKITCVETWPEKNPPPERQIPRRGEESSEMEQISIIERFPYP
FQVVYDEGPLYVFSPTEELRKRWIHQLKNVIRYNSDLVQKYHPCFWIDGQ
YLCCSQTAKNAMGCQILENRNGSLKP
Pleckstrin homology domain of Human BTK1
R28C:
(SEQ ID NO: 127)
MAAVILESIFLKRSQQKKKTSPLNFKKCLFLLTVHKLSYYEYDFERGRRG
SKKGSIDVEKITCVETWPEKNPPPERQIPRRGEESSEMEQISIIERFPYP
FQVVYDEGPLYVFSPTEELRKRWIHQLKNVIRYNSDLVQKYHPCFWIDGQ
YLCCSQTAKNAMGCQILENRNGSLKP
Pleckstrin homology domain of Human FAPP1
(SEQ ID NO: 128)
MEGVLYKWTNYLTGWQPRWFVLDNGILSYYDSQDDVCKGSKGSIKMAVCE
IKVHSADNTRMELIIPGEQHFYMKAVNAAERQRWLVALGSSKACLTDT
Pleckstrin homology domain of Human CERT
(SEQ ID NO: 129)
PVERCGVLSKWTNYIHGWQDRWVVLKNNALSYYKSEDETEYGCRGSICLS
KAVITPHDFDECRFDISVNDSVWYLRAQDPDHRQQWIDAIEQHKT
Pleckstrin homology domain of Human PHLPP1
(SEQ ID NO: 130)
MRIQLSGMYNVRKGKMQLPVNRWTRRQVILCGTCLIVSSVKDSLTGKMHV
LPLIGGKVEEVKKHQHCLAFSSSGPQSQTYYICFDTFTEYLRWLRQVSKV
AS
Pleckstrin homology domain of Human SWAP70
(SEQ ID NO: 131)
MDVLKQGYMMKKGHRRKNWTERWFVLKPNIISYYVSEDLKDKKGDILLDE
NCCVESLPDKDGKKCLFLVKCFDKTFEISASDKKKKQEWIQAIHSTIH
Pleckstrin homology domain of Human SWAP70
R223E, R224E:
(SEQ ID NO: 132)
MDVLKQGYMMKKGHEEKNWTERWFVLKPNIISYYVSEDLKDKKGDILLDE
NCCVESLPDKDGKKCLFLVKCFDKTFEISASDKKKKQEWIQAIHSTIH
Pleckstrin homology domain of Human MAPKAP1
(SEQ ID NO: 133)
MDMLSSHHYKSFKVSMIHRLRFTTDVQLGISGDKVEIDPVTNQKASTKFW
IKQKPISIDSDLLCACDLAEEKSPSHAIFKLTYLSNHDYKHLYFESDAAT
VNEIVLKVNYILES
Pleckstrin Homology Domain of Human PKD
(SEQ ID NO: 134)
MGTVMKEGWMVHYTSKDTLRKRHYWRLDSKCITLFQNDTGSRYYKEIPLS
EILSLEPVKTSALIPNGANPHCFEITTANVVYYVGENVVNPSSPSPNNSV
LTSGVGADVARMWEIAIQHALM
Pleckstrin homology domain of Human Son
Of Sevenless Homolog 2
(SEQ ID NO: 135)
FIMEGPLTRIGAKHERHIFLFDGLMISCKPNHGQTRLPGYSSAEYRLKEK
FVMRKIQICDKEDTCEHKHAFELVSKDENSIIFAAKSAEEKNNWMAALIS
LHYRS
Pleckstrin homology domain of Human Dynamin
(SEQ ID NO: 136)
QGTNLPPSRQIVIRKGWLTISNIGIMKGGSKGYWFVLTAESLSWYKDDEE
KEKKYMLPLDNLKVRDVEKSFMSSKHIFALFNTEQRNVYKDYRFLELACD
SQEDVDS
Pleckstrin homology domain of Human BCR
(SEQ ID NO: 137)
QLLKDSFMVELVEGARKLRHVFLFTDLLLCTKLKKQSGGKTQQYDCKWYI
PLTDLSFQMVDELEAVPNIPLVPDEELDALKIKISQIKNDIQREKRANKG
SKATERLKKKLSEQESLLLLMSPSMAFRVHSRNGKSYTFLISSDYERAEW
RENIREQQK
Pleckstrin homology domain of Human DBS
(SEQ ID NO: 138)
KLLMQGSFSVWTDHKRGHTKVKELARFKPMQRHLFLHEKAVLFCKKREEN
GEGYEKAPSYSYKQSLNMAAVGITENVKGDAKKFEIWYNAREEVYIVQAP
TPEIKAAWVNEIRKVLT
Pleckstrin homology domain of Homo sapiens
phospholipase Cδ1 (hPLCδ1)
(SEQ ID NO: 139)
MDSGRDFLTLHGLQDDEDLQALLKGSQLLKVKSSSWRRERFYKLQEDCKT
IWQESRKVMRTPESQLFSIEDIQEVRMGHRTEGLEKFARDVPEDRCFSIV
FKDQRNTLDLIAPSPADAQHWVLGLHKIIHHSGSMDQRQKLQHWIHSCLR
KADKNKDNKMSFKELQNFLKELNIQ
Pleckstrin homology domain of Homo sapiens
phospholipase Cδ1 (hPLCδ1) R40L:
(SEQ ID NO: 140)
MDSGRDFLTLHGLQDDEDLQALLKGSQLLKVKSTSWRRELFYKLQEDCKT
IWQESRKVMRTPESQLFSIEDIQEVRMGHRTEGLEKFARDVPEDRCFSIV
FKDQRNTLDLIAPSPADAQHWVLGLHKIIHHSGSMDQRQKLQHWIHSCLR
KADKNKDNKMSFKELQNFLK
Pleckstrin homology domain of Homo sapiens
AKT1 (hAKT)
(SEQ ID NO: 141)
MSDVAIVKEGWLHKRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) E17K:
(SEQ ID NO: 142)
MSDVAIVKEGWLHKRGKYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) K14R:
(SEQ ID NO: 143)
MSDVAIVKEGWLHRRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) K8R:
(SEQ ID NO: 144)
MSDVAIVREGWLHKRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T72A:
(SEQ ID NO: 145)
MSDVAIVKEGWLHKRGEYIKTWRPRYFLLKNDGTFIGYKERPQDVDQREA
PLNNFSVAQCQLMKTERPRPNAFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T92A:
(SEQ ID NO: 146)
MSDVAIVKEGWLHKRGEYIKTWRPRYFLLKNDGTFIGYKERPQDVDQREA
PLNNFSVAQCQLMKTERPRPNTFIIRCLQWTTVIERTFHVEAPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) R25C:
(SEQ ID NO: 147)
MSDVAIVKEGWLHKRGEYIKTWRPCYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T34D:
(SEQ ID NO: 148)
MSDVAIVKEGWLHKRGEYIKTWRPRYFLLKNDGDFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T34F:
(SEQ ID NO: 149)
MSDVAIVKEGWLHKRGEYIKTWRPRYFLLKNDGFFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T34L:
(SEQ ID NO: 150)
MSDVAIVKEGWLHKRGEYIKTWRPRYFLLKNDGLFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) T81Y:
(SEQ ID NO: 151)
MSDVAIVKEGWLHKRGEYIKTWRPRYFLLKNDGTFIGYKERPQDVDQREA
PLNNFSVAQCQLMKTERPRPNTFIIRCLQWYTVIERTFHVETPEEREEWT
TAIQTVADGLKKQEEEEMDFRSGSPSDNSGAEEMEVSLAKPKHRVTMNEF
EYLKLLGKGTFGKVDPPV
Pleckstrin homology domain of Homo sapiens
AKT1 (hAKT) K142A, H143A, R144A:
(SEQ ID NO: 152)
MSDVAIVKEGWLHKRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPAAAVTMNEF
EYLKLLGKGTFGKVDPPV
Pleckstrin homology domain of Homo sapiens
AKT1 (hAKT) T101C:
(SEQ ID NO: 153)
MSDVAIVKEGWLHKRGEYIKTWRPRYFLLKNDGTFIGYKERPQDVDQREA
PLNNFSVAQCQLMKTERPRPNTFIIRCLQWTTVIERTFHVETPEEREEWT
CAIQTVADGLKKQEEEEMDFRSGSPSDNSGAEEMEVSLAKPKHRVTMNEF
EYLKLLGKGTFGKVDPPV
Pleckstrin homology domain of Homo sapiens
PDPK1 (hPDPK1)
(SEQ ID NO: 154)
KMGPVDKRKGLFARRRQLLLTEGPHLYYVDPVNKVLKGEIPWSQELRPEA
KNFKTFFVHTPNRTYYLMDPSGNAHKWCRKIQEVWRQRYQSH
MS2 (RNA Binding protein):
(SEQ ID NO: 155)
MASNFTQFVLVDNGGTGDVTVAPSNFANGIAEWISSNSRSQAYKVTCSVR
QSSAQKRKYTIKVEVPKVATQTVGGVELPVAAWRSYLNMELTIPIFATNS
DCELIVKAMQGLLKDGNPIPSAIAANSGIY
COM (RNA Binding protein):
(SEQ ID NO: 156)
MKSIRCKNONKLLFKADSFDHIEIRCPRCKRHIIMLNACEHPTEKHCGKR
EKITHSDETVRY
PP7 (RNA Binding protein):
(SEQ ID NO: 157)
MAKTIVLAVGEATRTLTEIQSTADRQIFEEKVGPLVGRLRLTASLRQNGA
KTAYRVNLKLDQADVVDASTSVAGELPKVRYTQVWSHDVTIVANSTEASR
KSLYDLTKSLVATSQVEDLVVNLVPLGRSLE
TBP (RNA Binding protein):
(SEQ ID NO: 158)
MAVPETRPNHTIYINNLNSKIKKDELKKSLYAIFSQFGQILDILVPRQRT
PRGQAFVIFKEVSSATNALRSMQGFPFYDKPMRIQYAKTDKRIPAKMKGT
FV
Human SLBP (RNA Binding protein):
(SEQ ID NO: 159)
MADFETDESVLMRRQKQINYGKNTIAYDRYIKEVPRHLRQPGIHPKTPNK
FKKYSRRSWDQQIKLWKVALHFWD
Herpes simplex virus (HSV) type 1:
VP16 Transcription Activation Domain
(SEQ ID NO: 160)
PTDALDDFDLDMLPADALDDFDLDMLPADALDDFDLDM
Herpes simplex virus (HSV) type 1 &
Synthetic: VP64
(SEQ ID NO: 161)
GRADALDDFDLDMLGSDALDDFDLDMLGSDALDDFDLDMLGSDALDDFDL
DML
Homo sapiens: P65
(SEQ ID NO: 162)
SQYLPDTDDRHRIEEKRKRTYETFKSIMKKSPFSGPTDPRPPPRRIAVPS
RSSASVPKPAPQPYPFTSSLSTINYDEFPTMVFPSGQISQASALAPAPPQ
VLPQAPAPAPAPAMVSALAQAPAPVPVLAPGPPQAVAPPAPKPTQAGEGT
LSEALLQLQFDDEDLGALLGNSTDPAVFTDLASVDNSEFQQLLNQGIPVA
PHTTEPMLMEYPEAITRLVTGAQRPPDPAPAPLGAPGLPNGLLSGDEDFS
SIADMDFSALL
Kaposi's Sarcoma-Associated Herpesvirus
Transactivator: RTA
(SEQ ID NO: 163)
RDSREGMFLPKPEAGSAISDVFEGREVCQPKRIRPFHPPGSPWANRPLPA
SLAPTPTGPVHEPVGSLTPAPVPQPLDPAPAVTPEASHLLEDPDEETSQA
VKALREMADTVIPQKEEAAICGQMDLSHPPPRGHLDELTTTLESMTEDLN
LDSPLTPELNEILDTFLNDECLLHAMHISTGLSIFDTSLF
Homo sapiens: KRAB
(SEQ ID NO: 164)
MDAKSLTAWSRTLVTFKDVFVDFTREEWKLLDTAQQIVYRNVMLENYKNL
VSLGYQLTKPDVILRLEKGEEP
Homo sapiens: MeCP2
(SEQ ID NO: 165)
EASVQVKRVLEKSPGKLLVKMPFQASPGGKGEGGGATTSAQVMVIKRPGR
KRKAEADPQAIPKKRGRKPGSVVAAAAAEAKKKAVKESSIRSVQETVLPI
KKRKTRETVSIEVKEVVKPLLVSTLGEKSGKGLKTCKSPGRKSKESSPKG
RSSSASSPPKKEHHHHHHHAESPKAPMPLLPPPPPPEPQSSEDPISPPEP
QDLSSSICKEEKMPRAGSLESDGCPKEPAKTQPMVAAAATTTTTTTTTVA
EKYKHRGEGERKDIVSSSMPRPNREEPVDSRTPVTERVS
Homo sapiens: TET1
(SEQ ID NO: 166)
LPTCSCLDRVIQKDKGPYYTHLGAGPSVAAVREIMENRYGQKGNAIRIEI
VVYTGKEGKSSHGCPIAKWVLRRSSDEEKVLCLVRQRTGHHCPTAVMVVL
IMVWDGIPLPMADRLYTELTENLKSYNGHPTDRRCTLNENRTCTCQGIDP
ETCGASFSFGCSWSMYFNGCKFGRSPSPRRFRIDPSSPLHEKNLEDNLQS
LATRLAPIYKQYAPVAYQNQVEYENVARECRLGSKEGRPFSGVTACLDFC
AHPHRDIHNMNNGSTVVCTLTREDNRSLGVIPQDEQLHVLPLYKLSDTDE
FGSKEGMEAKIKSGAIEVLAPRRKKRTCFTQPVPRSGKKRAAMMTEVLAH
KIRAVEKKPIPRIKRKNNSTTTNNSKPSSLPTLGSNTETVQPEVKSETEP
HFILKSSDNTKTYSLMPSAPHPVKEASPGFSWSPKTASATPAPLKNDATA
SCGFSERSSTPHCTMPSGRLSGANAAAADGPGISQLGEVAPLPTLSAPVM
EPLINSEPSTGVTEPLTPHQPNHQPSFLTSPQDLASSPMEEDEQHSEADE
PPSDEPLSDDPLSPAEEKLPHIDEYWSDSEHIFLDANIGGVAIAPAHGSV
LIECARRELHATTPVEHPNRNHPTRLSLVFYQHKNLNKPQHGFELNKIKF
EAKEAKNKKMKASEQKDQAANEGPEQSSEVNELNQIPSHKALTLTHDNVV
TVSPYALTHVAGPYNHWV
Homo sapiens: DNMT3A
(SEQ ID NO: 167)
MPAMPSSGPGDTSSSAAEREEDRKDGEEQEEPRGKEERQEPSTTARKVGR
PGRKRKHPPVESGDTPKDPAVISKSPSMAQDSGASELLPNGDLEKRSEPQ
PEEGSPAGGQKGGAPAEGEGAAETLPEASRAVENGCCTPKEGRGAPAEAG
KEQKETNIESMKMEGSRGRLRGGLGWESSLRQRPMPRLTFQAGDPYYISK
RKRDEWLARWKREAEKKAKVIAGMNAVEENQGPGESQKVEEASPPAVQQP
TDPASPTVATTPEPVGSDAGDKNATKAGDDEPEYEDGRGFGIGELVWGKL
RGFSWWPGRIVSWWMTGRSRAAEGTRWVMWFGDGKFSVVCVEKLMPLSSF
CSAFHQATYNKQPMYRKAIYEVLQVASSRAGKLFPVCHDSDESDTAKAVE
VQNKPMIEWALGGFQPSGPKGLEPPEEEKNPYKEVYTDMWVEPEAAAYAP
PPPAKKPRKSTAEKPKVKEIIDERTRERLVYEVRQKCRNIEDICISCGSL
NVTLEHPLFVGGMCQNCKNCFLECAYQYDDDGYQSYCTICCGGREVLMCG
NNNCCRCFCVECVDLLVGPGAAQAAIKEDPWNCYMCGHKGTYGLLRRRED
WPSRLQMFFANNHDQEFDPPKVYPPVPAEKRKPIRVLSLFDGIATGLLVL
KDLGIQVDRYIASEVCEDSITVGMVRHQGKIMYVGDVRSVTQKHIQEWGP
FDLVIGGSPCNDLSIVNPARKGLYEGTGRLFFEFYRLLHDARPKEGDDRP
FFWLFENVVAMGVSDKRDISRFLESNPVMIDAKEVSAAHRARYFWGNLPG
MNRPLASTVNDKLELQECLEHGRIAKFSKVRTITTRSNSIKQGKDQHFPV
FMNEKEDILWCTEMERVFGFPVHYTDVSNMSRLARQRLLGRSWSVPVIRH
LFAPLKEYFACV
BaEVTRless
(SEQ ID NO: 168)
MGFTTKIIFLYNLVLVYAGFDDPRKAIELVQKRYGRPCDCSGGQVSEPPS
DRVSQVTCSGKTAYLMPDQRWKCKSIPKDTSPSGPLQECPCNSYQSSVHS
SCYTSYQQCRSGNKTYYTATLLKTQTGGTSDVQVLGSTNKLIQSPCNGIK
GQSICWSTTAPIHVSDGGGPLDTTRIKSVQRKLEEIHKALYPELQYHPLA
IPKVRDNLMVDAQTLNILNATYNLLLMSNTSLVDDCWLCLKLGPPTPLAI
PNFLLSYVTRSSDNISCLIIPPLLVQPMQFSNSSCLFSPSYNSTEEIDLG
HVAFSNCTSITNVTGPICAVNGSVFLCGNNMAYTYLPTNWTGLCVLATLL
PDIDIIPGDEPVPIPAIDHFIYRPKRAIQFIPLLAGLGITAAFTTGATGL
GVSVTQYTKLSNQLISDVQILSSTIQDLQDQVDSLAEVVLQNRRGLDLLT
AEQGGICLALQEKCCFYVNKSGIVRDKIKTLQEELERRRKDLASNPLWTG
LQGLLPYLLPFLGPLLTLLLLLTIGPCIFNRLTAFINDKLNIIHAM

### EXAMPLES

The invention is further described in the following examples, which do not limit the scope of the invention described in the claims.

### Example 1. Minimal Virus-Like Particles Deliver Gene Editing Cargo to Target Cells

### Methods

mVLP particles were produced in HEK293T cells by using polyethylenimine (PEI) to transfect plasmids into these cells. PEI is Polyethylenimine 25 kD linear (Polysciences #23966-2). To make a stock ‘PEI MAX’ solution, Ig of PEI was added to 1 L endotoxin-free dH₂O that was previously heated to ˜80° C. and cooled to room temperature. This mixture was neutralized to pH 7.1 by addition of 10N NaOH and filter sterilized with 0.22 m polyethersulfone (PES). PEI MAX was stored at −20° C.

HEK293T cells were split to reach a confluency of 70%-90% at time of transfection and are cultured in 10% FBS DMEM media. Plasmid vector encoding cargo, e.g., one encoding a CMV promoter driving expression of a hPLCS1 PH domain or another PH domain as shown herein fused to codon optimized Cas9 were co-transfected with a plasmids encoding a U6 promoter driving expression of a sgRNA and the VSV-G envelope plasmid. Transfection reactions were assembled in reduced serum media (Opti-MEM; GIBCO #31985-070). For mVLP particle production on 10 cm plates, 7.5 μg PH-Cas9 expressing plasmid, 7.5 μg sgRNA-expression plasmid and 5 μg VSVG expressing plasmid were mixed in 1 mL Opti-MEM, followed by addition of 27.5 μl PEI MAX. After 20-30 min incubation at room temperature, the transfection reactions were dispersed dropwise over the HEK293T cells.

mVLPs were harvested at 48-72 hours post-transfection. To do this, mVLP supernatants were filtered using 0.45 μm PVDF or cellulose acetate or 0.8 μm PES membrane filters and transferred to polypropylene Beckman ultracentrifuge tubes that are used with the SW28 rotor (Beckman Coulter #326823). Each ultracentrifuge tube is filled with mVLP-containing supernatant from three 10 cm plates to reach an approximate final volume of 35-37.5 ml. mVLP supernatant underwent ultracentrifugation at approximately 100,000×g, or 25,000 rpm, at 4° C. for 2 hours. After ultracentrifugation, supernatants were decanted and mVLP pellets resuspended in DMEM 10% FBS media, or other media appropriate for the culturing of recipient cells, such that they are now approximately 1,000 times more concentrated than they were before ultracentrifugation. mVLPs were added dropwise to cells that were seeded in a 24-well plate 24-hours prior to transduction. Polybrene (5-10 μg/mL in cell culture medium; Sigma-Aldrich #TR-1003-G) was supplemented to enhance transduction efficiency, if necessary. Vectofusin-1 (10 μg/mL in cell culture medium, Miltenyi Biotec #130-111-163) was supplemented to enhance transduction efficiency, if necessary. Immediately following the addition of mVLPs, the 24-well plate was centrifuged at 1,150×g for 30 min at room temperature to enhance transduction efficiency, if necessary.

### Example 1.1

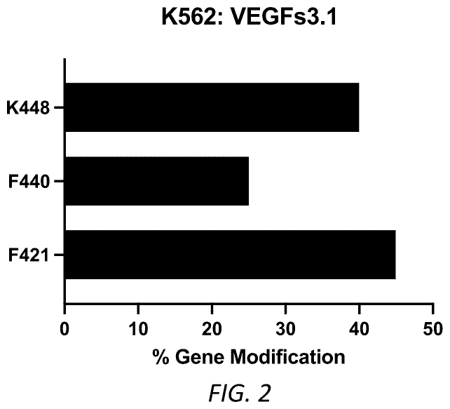

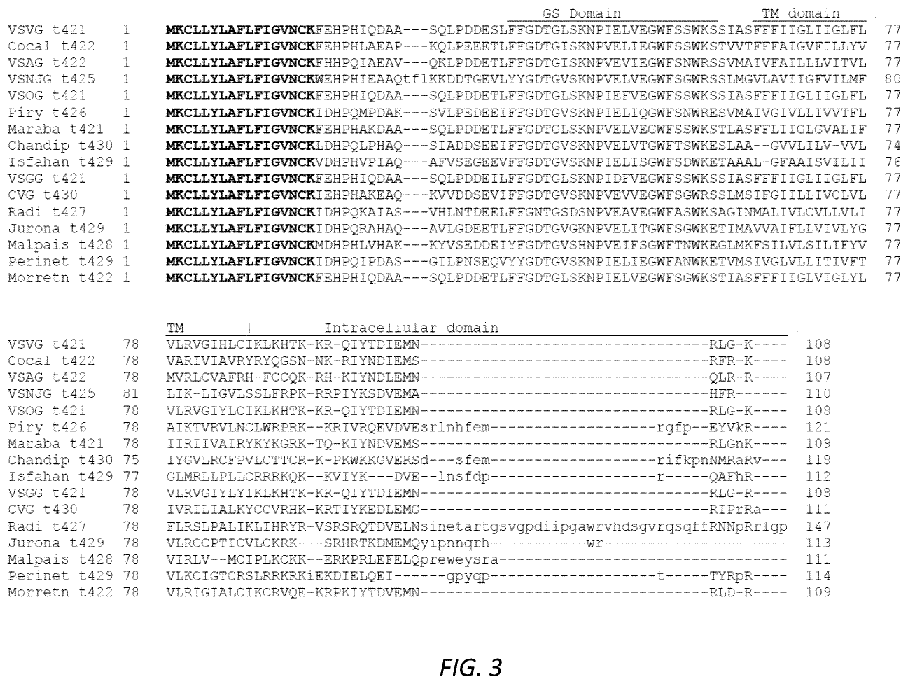

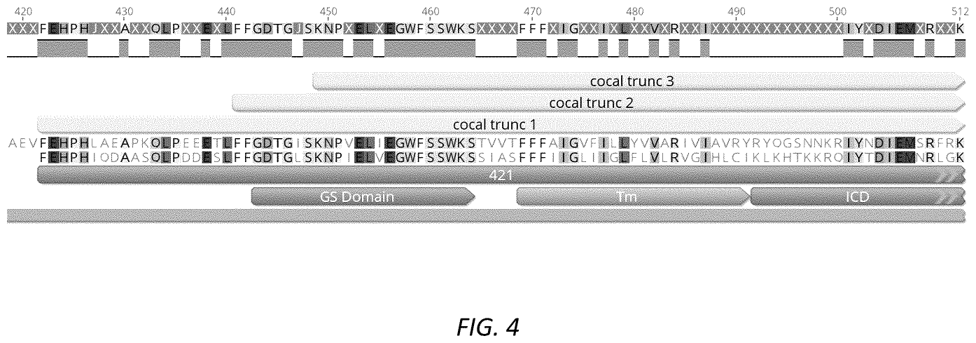

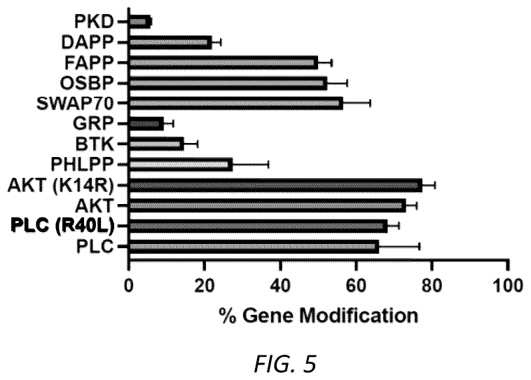

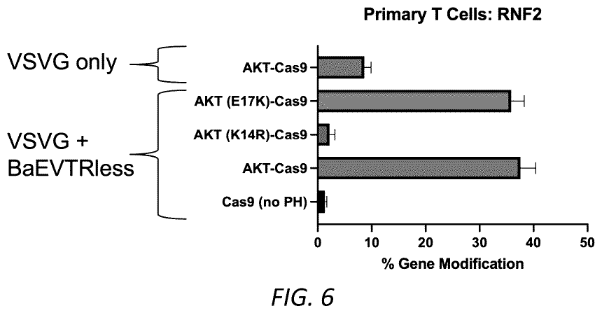

mVLPs were produced by transient plasmid transfection of HEK293T cells (FIG. 1A). mVLPs were purified and concentrated 100-fold by filtration and PEG precipitation (FIG. 1B) and applied to K562 cells for an incubation period of 48 hours. K562 cells were harvested and genomic DNA was extracted. Targeted amplicon sequencing of extracted genomic DNA was used to quantify frequencies of gene modifications (y-axis) (i.e., gene edits) at the intended VEGFs3.1 on-target site (FIG. 2). We observed high frequencies of gene modification at this intended target sequence in K562 cells, demonstrating that mVLPs are capable of efficiently delivering RNP CRISPR-based cargo into living mammalian cells. FIGS. 3 and 4 are exemplary alignments of tENVs that are described herein.

### Example 1.2

FIGS. 5-6 show that different phospholipid bilayer recruitment domains are capable of delivering cargo in previously described eVLPs (WO 2022/020800). eVLPs were produced by transient transfection of HEK293T cells, purified and concentrated 100-fold by filtration and PEG precipitation, and normalized based on Cas9 ELISA prior to transducing HEK293T cells so that the same pmol of Cas9 was applied in each well and comparisons could be made between different PH domains. Percent editing of endogenous VEGF was determined by amplicon sequencing (FIG. 5). These eVLPs were pseudotyped with VSVG. The results showed that different PH domain and mutant PH domain fusions to cargos will result in different delivery efficiencies.

Different mutant PH-Cas9 fusions (and Cas9 lacking a fusion to a PH domain) were packaged in eVLPs (made as described in WO 2022/020800), purified and concentrated 100-fold by PEG precipitation, and normalized by Cas9 ELISA so that 5 pmol of Cas9 was added to 15,000 primary T cells per well. Percent editing of endogenous RNF2 was determined by amplicon sequencing (FIG. 6). These eVLPs were pseudotyped with VSVG or a combination of VSVG and BaEVTRless. The results showed that different PH domain and mutant PH domain fusions to cargos will result in different delivery efficiencies which could be cell type dependent. In addition, different pseudotype combinations can also increase or decrease delivery efficiency.

### REFERENCES

1. Parseval, N. et al. Survey of human genes of retroviral origin: identification and transcriptome of the genes with coding capacity for complete envelope proteins. Journal of Virology 77, 10414-10422, (2003).
2. Okimoto, T. et al. VSV-G envelope glycoprotein forms complexes with plasmid DNA and MLV retrovirus-like particles in cell-free conditions and enhances DNA transfection. Molecular Therapy 4, 232-238, (2001).
3. Mangeot, P. et al. Protein transfer into human cells by VSV-G-induced nanovesicles. Molecular Therapy 19, 1656-1666, (2011).
4. Wagner, D. et al. High prevalence of Streptococcus pyogenes Cas9-reactive T cells within the adult human population. Nature Medicine 25, 242-248 (2019)
5. Kim, S. et al. CRISPR RNAs trigger innate immune responses in human cells. Genome Research 28, 1-7 (2018).
6. Charlesworth, C. et al. Identification of preexisting adaptive immunity to Cas9 proteins in humans. Nature Medicine 25, 249-254 (2019)
7. Ferdosi, S. et al. Multifunctional CRISPR-Cas9 with engineered immunosilenced human T cell epitopes. Nature Communications 10, Article number: 1842 (2019).
8. Wang, D. et al. Adenovirus-mediated somatic genome editing of Pten by CRISPR/Cas9 in mouse liver in spite of Cas9-specific immune responses. Human Gene Therapy 26, 432-442 (2015).
9. Devanabanda, M. et al. Immunotoxic effects of gold and silver nanoparticles: Inhibition of mitogen-induced proliferative responses and viability of human and murine lymphocytes in vitro. Journal of Immunotoxicology 13, 1547-6901 (2016).
10. Mout, R. et al. Direct cytosolic delivery of CRISPR/Cas9-ribonucleoprotein for efficient gene editing. ACS Nano 11, 2452-2458 (2017).
11. Yin, H. et al. structure-guided chemical modification of guide RNA enables potent non-viral in vivo genome editing. Nature Biotechnology 35, 1179-1187 (2017).
12. Qiao, J. et al. Cytosolic delivery of CRISPR/Cas9 ribonucleoproteins for genome editing using chitosan-coated red fluorescent protein. Chemical Communications 55, 4707-4710 (2019).
13. Li, L. et al. A rationally designed semiconducting polymer brush for NIR-II imaging guided light-triggered remote control of CRISPR/Cas9 genome editing. Advanced Materials 1901187, 1-9 (2019).
14. Gao, X. et al. Treatment of autosomal dominant hearing loss by in vivo delivery of genome editing agents. Nature 553, 217-221 (2018)
15. Lee, K. et al. Nanoparticle delivery of Cas9 ribonucleoprotein and donor DNA in vivo induces homology-directed DNA repair. Nature Biomedical Engineering 1, 889-901 (2017).
16. Staahl, B. et al. Efficient genome editing in the mouse brain by local delivery of engineered Cas9 ribonucleoprotein complexes. Nature Biotechnology 35, 431-433 (2017).
17. Zuris, J. et al. Cationic lipid-mediated delivery of proteins enables efficient protein-based genome editing in vitro and in vivo. Nature Biotechnology 33, 73-79 (2015).
18. Finn, J. et al. A single administration of CRISPR/Cas9 lipid nanoparticles achieves robust and persistent in vivo genome editing. Cell Reports 22, 2227-2235 (2018).
19. Wang, H. et al. Nonviral gene editing via CRISPR/Cas9 delivery by membrane-disruptive and endosomolytic helical polypeptide. PNAS 115, 4903-4908 (2018).
20. Del'Guidice, T. et al. Membrane permeabilizing amphiphilic peptide delivers recombinant transcription factor and CRISPR-Cas9/Cpf1 ribonucleoproteins in hard-to-modify cells. PLOS ONE 13, e0195558 (2018).
21. Colella, P. et al. Emerging Issues in AAV-Mediated In Vivo Gene Therapy. Molecular Therapy: Methods & Clinical Development 8, 87-104 (2018).
22. Naso, F. et al. Adeno-Associated Virus (AAV) as a Vector for Gene Therapy. BioDrugs 31, 317-334 (2017).
23. Handel, E. et al. Versatile and efficient genome editing in human cells by combining zinc-finger nucleases with adeno-associated viral vectors. Human Gene Therapy 23, 321-329 (2012).
24. Chadwick, A. et al. Reduced Blood Lipid Levels With In Vivo CRISPR-Cas9 Base Editing of ANGPTL3. Circulation 137, 975-977 (2018).
25. Schenkwein, D. et al. Production of HIV-1 Integrase Fusion Protein-Carrying Lentiviral Vectors for Gene Therapy and Protein Transduction. Human Gene Therapy 21, 589-602 (2010).
26. Cai, Y. et al. Targeted genome editing by lentiviral protein transduction of zinc-finger and TAL-effector nucleases. eLife 3, e01911 (2014).
27. Choi, J. et al. Lentivirus pre-packed with Cas9 protein for safer gene editing. Gene Therapy 23, 627-633 (2016).
28. Meyer, C. et al. Pseudotyping exosomes for enhanced protein delivery in mammalian cells. International Journal of Nanomedicine 12, 3153-3170 (2017).
29. Mangeot, P. et al. Genome editing in primary cells and in vivo using viral-derived Nanoblades loaded with Cas9-sgRNA ribonucleoproteins. Nature Communications 10, Article number: 45 (2019).
30. Lu, B. et al. Delivering SaCas9 mRNA by lentivirus-like bionanoparticles for transient expression and efficient genome editing. Nucleic Acids Research 47, e44 (2019).
31. Wang, Q. et al. ARMMs as a versatile platform for intracellular delivery of macromolecules. Nature Communications 9, 1-7 (2018).
32. Lainscek, D. et al. Delivery of an Artificial Transcription Regulator dCas9-VPR by Extracellular Vesicles for Therapeutic Gene Activation. ACS Synthetic Biology 7, 2715-2725 (2018).
33. Fuchs, J. et al. First-in-Human Evaluation of the Safety and Immunogenicity of a Recombinant Vesicular Stomatitis Virus Human Immunodeficiency Virus-1 gag Vaccine (HVTN 090). Open Forum Infectious Diseases 2, 1-9, (2015).
34. Cong, L. et al. Multiplex Genome Engineering Using CRISPR/Cas Systems. Science 339, 819-823, (2013).
35. Ran, F. et al. In vivo genome editing using Staphylococcus aureus Cas9. Nature 520, 186-191, (2015).
36. Zetsche, B. et al. Cpf1 Is a Single RNA-Guided Endonuclease of a Class 2 CRISPR-Cas System. Cell 163, 759-771, (2015).
37. Komor, A. et al. Programmable editing of a target base in genomic DNA without double-stranded DNA cleavage. Nature 533, 420-424, (2016).
38. Gaudelli, N. et al. Programmable base editing of A·T to G·C in genomic DNA without DNA cleavage. Nature 551, 464-471, (2017).
39. Voelkel, C. et al. Protein transduction from retroviral Gag precursors. Proc Natl Acad Sci USA 107, 7805-7810, (2010).
40. Kaczmarczyk, S. et al. Protein delivery using engineered virus-like particles. Proc Natl Acad Sci USA 108, 16998-17003, (2011).
41. Ebner, M. et al. PI(3,4,5)P₃ Engagement Restricts Akt Activity to Cellular Membranes. Mol Cell 65, 416-431, (2017).
42. Urano, E. et al. Substitution of the myristoylation signal of human immunodeficiency virus type 1 Pr55Gag with the phospholipase C-dl pleckstrin homology domain results in infectious pseudovirion production. J. Gen Virology 89, 3144-3149, (2008).
43. Pastuzyn, E. et al. The Neuronal Gene Arc Encodes a Repurposed Retrotransposon Gag Protein that Mediates Intercellular RNA Transfer. Cell 172, 275-288, (2018).
44. Lukacs, G. et al. Size-dependent DNA Mobility in Cytoplasm and Nucleus. Journal of Biological Chemistry 275, 1625-1629, (1999).
45. Kreiss, P. et al. Plasmid DNA size does not affect the physicochemical properties of lipoplexes but modulates gene transfer efficiency. Nucleic Acids Research 27, 3792-3798 (1999).
46. Nafissi, N. et al. DNA Ministrings: Highly Safe and Effective Gene Delivery Vectors. Molecular Therapy-Nucleic Acids 3, e165, (2014).
47. Fujimoto, T. et al. Selective EGLN Inhibition Enables Ablative Radiotherapy and Improves Survival in Unresectable Pancreatic Cancer. Cancer Research 79, 2327-2338 (2019).
48. Tai, S. et al. Differential Expression of Metallothionein 1 and 2 Isoforms in Breast Cancer Lines with Different Invasive Potential: Identification of a Novel Nonsilent Metallothionein-1H Mutant Variant. American Journal of Pathology 163, 2009-2019 (2003).
49. Caussinus, E. et al. Fluorescent fusion protein knockout mediated by anti-GFP nanobody. Nature Structural & Molecular Biology 19, 117-121, (2012).
50. Zhao, W. et al. Quantitatively Predictable Control of Cellular Protein Levels through Proteasomal Degradation. ACS Synthetic Biology 7, 540-552, (2018).
51. Clift, D. et al. A Method for the Acute and Rapid Degradation of Endogenous Proteins. Cell 171, 1692-1706, (2017).
52. Balla, T. & Virnai, T. Visualizing Cellular Phosphoinositide Pools with GFP-Fused Protein-Modules. SCIENCE'S STKE 2002, p. pl3, DOI: 10.1126/stke.2002.125.pl3 (2002).
53. Carpten, J. et al. A transforming mutation in the pleckstrin homology domain of AKT1 in cancer. Nature 448, 439-445, (2007).
54. Virnai, P. et al. Selective cellular effects of overexpressed pleckstrin-homology domains that recognize PtdIns(3,4,5)P3 suggest their interaction with protein binding partners. Journal of Cell Science 118, 4879-4888, (2005).
55. Parikh, C. et al. Disruption of PH-kinase domain interactions leads to oncogenic activation of AKT in human cancers. PNAS 109, 19368-19373, (2012).
56. Jo, H. et al. Small molecule-induced cytosolic activation of protein kinase Akt rescues ischemia-elicited neuronal death. PNAS 109 (26) 10581-10586, (2012).
57. Han, F. et al. The critical role of AMPK in driving Akt activation under stress, tumorigenesis and drug resistance. Nat Commun 9, 4728 (2018). doi.org/10.1038/s41467-018-07188-9
58. Li, X. et al. Autophosphorylation of Akt at Threonine 72 and Serine 246: A POTENTIAL MECHANISM OF REGULATION OF Akt KINASE ACTIVITY*. Journal of Biological Chemistry 281, 13837-13843, (2006).
59. Liao, Y. et al. Peptidyl-prolyl cis/trans isomerase Pin1 is critical for the regulation of PKB/Akt stability and activation phosphorylation. Oncogene 28(26):2436-45, DOI:10.1038/onc.2009.98 (2009).
60. Chu, N. et al. Akt Kinase Activation Mechanisms Revealed Using Protein Semisynthesis. Cell 174(4): 897-907.e14. (2018).
61. Lucid, I. et al. Conformational sampling of membranes by Akt controls its activation and inactivation. Proc Natl Acad Sci USA 115(17): E3940 E3949, (2018).
62. Baranov, M. et al. SWAP70 Organizes the Actin Cytoskeleton and Is Essential for Phagocyiosis. Cell Reports 17, p 1518-1531 (2016)

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A truncated glycoprotein/envelope protein (tENV) comprising:
an N-terminal portion comprising a signal sequence, optionally comprising the MKCLLYLAFLFIGVNCK (SEQ ID NO:1), fused to a central portion comprising all or part of a GS domain from at least one vesiculovirus G protein, optionally a VSV-G protein or homolog, ortholog, or paralog thereof, optionally comprising the sequence FEHPHIQDAASQLPDDESLFFGDTGLSKNPIELVEGWFSSWK (SEQ ID NO:2), optionally with a deletion of at least one amino acid (optionally a truncation from the N terminal end of the central portion), which is fused to a C-terminal portion comprising a transmembrane domain and an intracellular domain, optionally comprising
(SEQ ID NO: 3)
SSIASFFFIIGLIIGLFLVLRVGIHLCIKLKHTKKRQIYTDIEMNRLGK.

2. The tENV of claim 1, wherein the central portion comprises a deletion of at least 1, 2, 3, 4, 5, 6, 7, 8, 9, 10, 15, 20, 25, 30, 35, 38, 39, 40, 41, or all 42 amino acids of SEQ ID NO:2 amino acids, e.g., at least about 1, 2, 3, 4, 5, 6, 7, 8 or 10 amino acids, up to about 15, 20, 35, 30, 35, 38, 39, 40, 41, or all 42 amino acids, with any range therebetween.

3. The tENV of claim 1, wherein the central portion comprises
(SEQ ID NO: 4)
FFGDTGLSKNPIELVEGWFSSWK
or
(SEQ ID NO: 2)
FEHPHIQDAASQLPDDESLFFGDTGLSKNPIELVEGWFSSWK.

4. The tENV of claim 1, comprising a sequence that is at least 95% identical to a sequence set forth herein, e.g., in Table 1.

5. A nucleic acid sequence encoding the tENV of claim 1.

6. A vector comprising a nucleic acid sequence encoding the tENV of claim 1, optionally operably linked to a promoter for expression of the tENV of claim 1.

7. A host cell comprising the nucleic acid sequence encoding the tENV of claim 1, and optionally expressing the tENV of claim 1.

8. A virus-like particle (VLP) comprising the tENV of claim 1.

9. A minimal virus-like particle (mVLP), comprising:
a membrane comprising a phospholipid bilayer and the tENV of claim 1; and
optionally, a cargo disposed in the core of the mVLP, wherein the cargo is optionally fused to a phospholipid bilayer recruitment domain; and,
wherein the mVLP does not comprise an exogenous gag, pro, or pol protein.

10. The mVLP of claim 9, wherein the cargo is a therapeutic or diagnostic protein or nucleic acid encoding a therapeutic or diagnostic protein, or a chemical, optionally a small molecule therapeutic or diagnostic.

11. The mVLP of claim 9, wherein the cargo is a gene editing or epigenetic modulating reagent.

12. The mVLP of claim 9, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA.

13. The mVLP of claim 12, wherein the cargo is selected from the proteins listed in Tables 2, 3, 4 & 5, or that is at least 95% identical to a sequence set forth in Table 2, 3, 4, or 5.

14. The mVLP of claim 12, wherein the cargo comprises a CRISPR-Cas protein, and the mVLP further comprises one or more guide RNAs that bind to and direct the CRISPR-Cas protein to a target nucleic acid sequence.

15. The mVLP of claim 9, wherein the cargo comprises a fusion to a phospholipid bilayer recruitment domain, preferably as shown in Table 6, or that is at least 95% identical to a sequence set forth herein in Table 6.

16. A method of delivering a cargo to a target cell, optionally a cell in vivo or in vitro, the method comprising contacting the cell with the mVLP of claim 9 comprising the cargo.

17. A method of producing a VLP or an mVLP comprising a cargo, the method comprising:
providing a cell expressing the tENV of claim 1 and a cargo, optionally wherein the cell does not express an exogenous gag, pro, or pol protein; and
maintaining the cell under conditions such that the cells produce the VLPs or mVLPs.

18. The method of claim 17, further comprising harvesting and optionally purifying and/or concentrating the produced VLPs or mVLPs.

19. The method of claim 17, wherein the cargo is a therapeutic or diagnostic protein or nucleic acid encoding a therapeutic or diagnostic protein, or a small molecule, optionally a therapeutic or diagnostic small molecule.

20. The method of claim 17, wherein the cargo is a gene editing or epigenetic modulating reagent.

21. The method of claim 17, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA.

22. The method of claim 21, wherein the cargo reagent is selected from the proteins listed in Tables 2, 3, 4 & 5, or that is at least 95% identical to a sequence set forth in Table 2, 3, 4, or 5.

23. The method of claim 21, wherein the cargo reagent comprises a CRISPR-Cas protein, variant, or fusion thereof and the mVLP further comprises one or more guide RNAs that bind to and direct the CRISPR-based genome editing or modulating protein to a target sequence.

24. The method of claim 17, wherein the cargo comprises a fusion to a phospholipid bilayer recruitment domain, preferably as shown in Table 6, or that is at least 95% identical to a sequence set forth herein in Table 6.

25. A cell expressing the tENV of claim 1, and a cargo, optionally wherein the cell does not express an exogenous gag, pro, or pol protein.

26. The cell of claim 25, wherein the cargo is a therapeutic or diagnostic protein or nucleic acid encoding a therapeutic or diagnostic protein, or a small molecule, optionally a therapeutic or diagnostic small molecule.

27. The cell of claim 25, wherein the cargo is a gene editing or epigenetic modulating reagent.

28. The cell of claim 25, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA.

29. The cell of claim 28, wherein the cargo reagent is selected from the proteins listed in Tables 2, 3, 4, & 5, or that is at least 95% identical to a sequence set forth in Table 2, 3, 4, or 5.

30. The cell of claim 28, wherein the gene editing or epigenetic modulating reagent comprises a CRISPR-Cas protein, and the mVLP further comprises one or more guide RNAs that bind to and direct the CRISPR-Cas protein to a target sequence.

31. The cell of claim 25, wherein the cargo comprises a fusion to a phospholipid bilayer recruitment domain, preferably as shown in Table 6, or that is at least 95% identical to a sequence set forth herein in Table 6.

32. A primary or stable human cell line comprising cells expressing the tENV of claim 1, and a cargo, optionally wherein the cell does not express an exogenous gag, pro, or pol protein.

33. The cells of claim 32, which are Human Embryonic Kidney (HEK) 293 cells or HEK293 T cells.
